(19) United States
(12) Patent Application Publication
(10) Pub. No.: US 2023/0401303 A1
(43) Pub. Date: Dec. 14, 2023

(54) CONTACTLESS AUTHENTICATION SYSTEM AND AUTHENTICATION METHOD

(71) Applicant: Panasonic Intellectual Property Management Co., Ltd., Osaka (JP)

(72) Inventor: KATSUYA NOZAWA, Osaka (JP)

(21) Appl. No.: 18/454,244

(22) Filed: Aug. 23, 2023

Related U.S. Application Data
(63) Continuation of application No. PCT/JP2022/004157, filed on Feb. 3, 2022.

(30) Foreign Application Priority Data
Mar. 16, 2021 (JP) 2021-042210

(51) Int. Cl. G06F 21/32; G06V 10/141; G06V 40/12

(52) U.S. Cl. CPC G06F 21/32; G06V 10/141; G06V 40/1341; G06V 40/1371

(57) ABSTRACT

A contactless authentication system includes: at least one illumination apparatus that projects illumination light onto a portion of a hand, the portion being not in contact of an object, the illumination light containing a light component in a wavelength range greater than or equal to 1380 nm; and an imaging apparatus that obtains at least one selected from the group consisting of a fingerprint image and a palm print image as authentication information by imaging the light component in the wavelength range in reflected light generated by reflection of the illumination light from the portion of the hand.

### BACKGROUND

### 1. Technical Field

The present disclosure relates to a contactless authentication system and an authentication method.

### 2. Description of the Related Art

An act of shooting an image of a hand and extracting information that is characteristic of an individual from the shot image has been popularly practiced in order to authenticate the individual. The information being characteristic of the individual includes asperities constituting fingerprints and palm prints, distribution of sweat pores, and the like.

A general fingerprint authentication apparatus adopts a mode of pressing a finger against a glass surface of, for example, a prism as disclosed in Japanese Unexamined Patent Application Publication No. 7-334649, for example. In the case of this mode, light projected onto the finger is totally reflected at a recess in the finger not in contact with the glass surface. On the other hand, the total reflection of the light projected onto the finger disappears at a projection on the finger in contact with the glass surface. As a consequence, it is possible to obtain a fingerprint image at high contrast.

In the meantime, there is a growing demand for a contactless authentication technique that does not require the press of the finger and the like against the glass surface and the like from a hygiene perspective and from a need for authentication processing of a large number of people in a short time.

### SUMMARY

In one general aspect, the techniques disclosed here feature a contactless authentication system including: at least one illumination apparatus that projects illumination light onto a portion of a hand, the portion being not in contact of an object, the illumination light containing a light component in a wavelength range greater than or equal to 1380 nm; and an imaging apparatus that obtains at least one selected from the group consisting of a fingerprint image and a palm print image as authentication information by imaging the light component in the wavelength range in reflected light generated by reflection of the illumination light from the portion of the hand.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating a result of irradiating a finger with illumination light and shooting images thereof while using LEDs having central wavelengths different from one another;

FIG. 2 is a diagram illustrating emission spectra of the LEDs used for shooting the fingerprint images illustrated in FIG. 1;

FIG. 3 is a conceptual diagram illustrating paths of light projected onto a surface of a finger;

FIG. 4 is a diagram illustrating wavelength dependencies of intensities of subsurface scattering light when the light is made incident on the skin while changing wavelengths thereof;

FIG. 5 is a diagram illustrating a wavelength dependency of an absorption coefficient of water;

FIG. 6 is a block diagram illustrating a schematic configuration of a contactless authentication system according to Embodiment 1;

FIG. 7 is a sectional view illustrating an example of a schematic configuration of a photoelectric conversion element provided to an imaging element according to Embodiment 1;

FIG. 8 is a diagram illustrating a wavelength dependency of an intensity of solar light on a ground surface;

FIG. 9 is a flowchart illustrating an operation example of the contactless authentication system according to Embodiment 1;

FIG. 10 is a block diagram illustrating a schematic configuration of a contactless authentication system according to Embodiment 2;

FIG. 11 is a conceptual diagram of irradiation of a finger surface with illumination light;

FIG. 12 is a flowchart illustrating an operation example of the contactless authentication system according to Embodiment 2;

FIG. 13 is a block diagram illustrating a schematic configuration of a contactless authentication system according to a modified example of Embodiment 2;

FIG. 14 is a block diagram illustrating a schematic configuration of a contactless authentication system according to Embodiment 3;

FIG. 15 is a diagram illustrating examples of a change in emission intensity of illumination light and of a change in sensitivity of the imaging apparatus according to Embodiment 3; and

FIG. 16 is a flowchart illustrating an operation example of the contactless authentication system according to Embodiment 3.

### DETAILED DESCRIPTIONS

The authentication mode that does not bring the hand into contact with the glass surface and the like cannot use the presence of the total reflection as mentioned above. Accordingly, this mode has a difficulty in obtaining a high-contrast image. On the other hand, the use of low-contrast images as authentication information may cause authentication errors.

Given the circumstances, the present disclosure provides a contactless authentication system and the like which can obtain authentication information from a hand not in contact with an object so as to suppress the occurrence of an authentication error.

### Underlying Knowledge Forming Basis of Aspect of Present Disclosure

As described above, it is difficult to obtain a high-contrast fingerprint image from the image shot without bringing the hand into contact with the glass surface and the like. For this reason, it is more likely to cause an authentication error when a fingerprint image obtained in a non-contact manner is used as authentication information. Given the circumstances, the inventor has repeatedly carried out tests for imaging fingerprints by using illumination light at various wavelengths, thus having obtained the following knowledge.

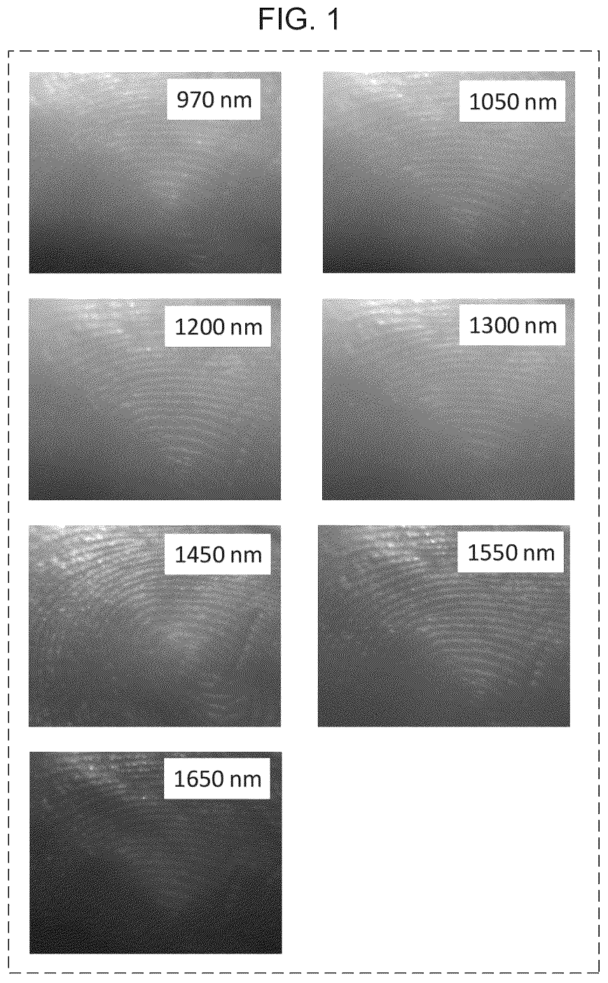

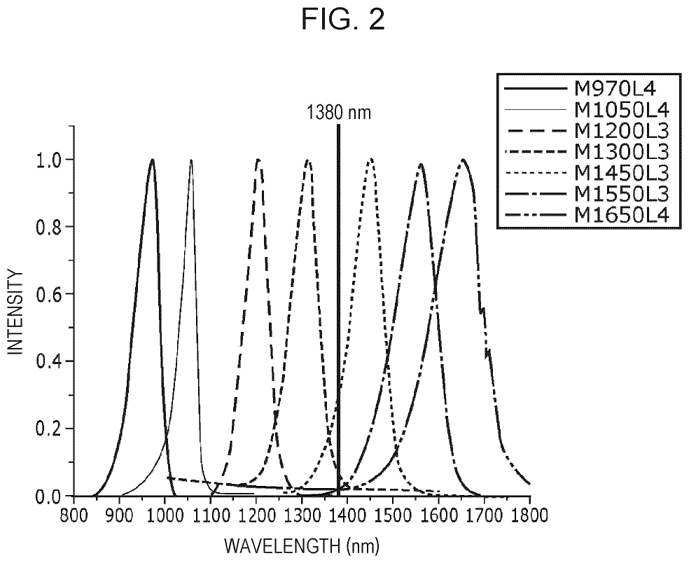

FIG. 1 is a diagram illustrating a result of irradiating a finger with illumination light and shooting images thereof while using light emitting diodes (LEDs) manufactured by Thorlabs, Inc. which have central wavelengths different from one another. FIG. 1 illustrates images of fingerprints that represent the result of shooting the images by using the LEDs having the central wavelengths of light emission of 970, 1050, 1200, 1300, 1450, 1550, and 1650 nm. Numerical values affixed to the respective fingerprint images in FIG. 1 represent the central wavelengths of the LEDs. In the meantime, FIG. 2 is a diagram illustrating emission spectra of the LEDs used for shooting the fingerprint images illustrated in FIG. 1, which is provided by Thorlabs, Inc. for reference. In shooting each fingerprint image illustrated in FIG. 1, the illumination light was projected from diagonally forward of the fingerprint on the finger, and the image was shot from the front of the finger.

As illustrated in FIG. 1, each fingerprint image has low contrast when any of the LEDs having the central wavelengths of 970, 1050, 1200, and 1300 nm is used. On the other hand, each fingerprint image has high contrast when any of the LEDs having the central wavelengths of 1450, 1550, and 1650 nm is used. In other words, the fingerprint images are clearly shot in these cases. Moreover, an image of sweat pores being pores through which sweat comes out is clearly shot in addition to the high contrast of the fingerprint image when any of the LEDs having the central wavelengths of 1450, 1550, and 1650 nm is used. Specifically, white dots in the fingerprint image are the sweat pores. In addition, images of wrinkles on the skin are also clearly shot as with the fingerprints when any of the LEDs having the central wavelengths of 1450, 1550, and 1650 nm is used.

Similar imaging results are also obtained in a test of using a halogen lamp including a wide wavelength range of illumination light instead of the above-described LEDs, attaching a bandpass filter to transmit light at specific wavelengths to an imaging apparatus, changing the wavelength to transmit the bandpass filter, and imaging the light reflected from a finger and transmitted through the bandpass filter.

Moreover, the inventor has investigated a cause of the aforementioned difference in contrast or the like, and has found out that a component of scatter-reflected light called subsurface scattering light that enters the skin and is emitted again therefrom is the cause of the aforementioned phenomenon.

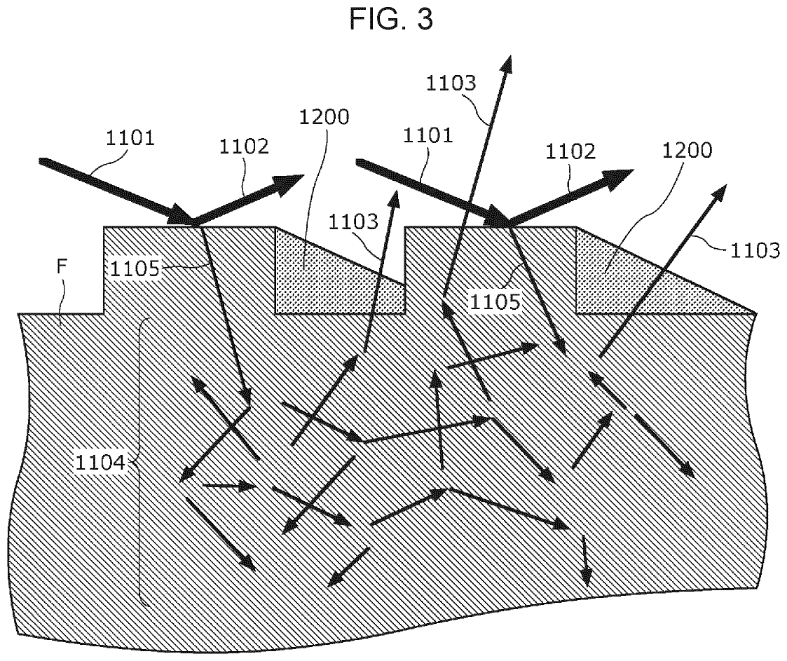

FIG. 3 is a conceptual diagram illustrating paths of light projected onto a surface of a finger.

As illustrated in FIG. 3, part of light 1101 projected onto a surface of a finger F is reflected from the surface and changed into surface-reflected light 1102. The surface-reflected light 1102 is increased at a projection that is exposed more to the light 1101 and is decreased at a recess 1200 shaded by the projection. Accordingly, a component of the surface-reflected light 1102 includes a lot of information concerning a fingerprint that represents information on asperities on the finger.

Meanwhile, another part of the light 1101 projected onto the surface of the finger F enters the finger F. Such light 1105 entering the finger F is scattered many times and spreads into the finger, thereby constituting scattered light 1104 traveling in various directions. Part of the scattered light 1104 is emitted again from the surface of the finger F. This light emitted again from the surface of the finger F is also referred to as subsurface scattering light 1103. The subsurface scattering light 1103 is the scatter-reflected light from the finger F which originates from the light 1101. The subsurface scattering light 1103 is the light that has lost information on the surface of the finger F on which the light was made incident in the first place as a consequence of the scattering inside the finger F. Moreover, the subsurface scattering light 1103 is emitted almost in the same way from the projection and from the recess of the finger F. Accordingly, the subsurface scattering light 1103 contains very little information on the fingerprint being the information on the asperities on the finger unlike the surface-reflected light 1102.

Due to the optical paths as described above, an image of a fingerprint on a finger in a state of non-contact with the glass surface and the like is shot more clearly when there are more components of the surface-reflected light 1102 or shot more vaguely when there are more components of the subsurface scattering light 1103.

Next, the inventor has carried out the following test in order to investigate wavelength dependencies of intensities of the subsurface scattering light.

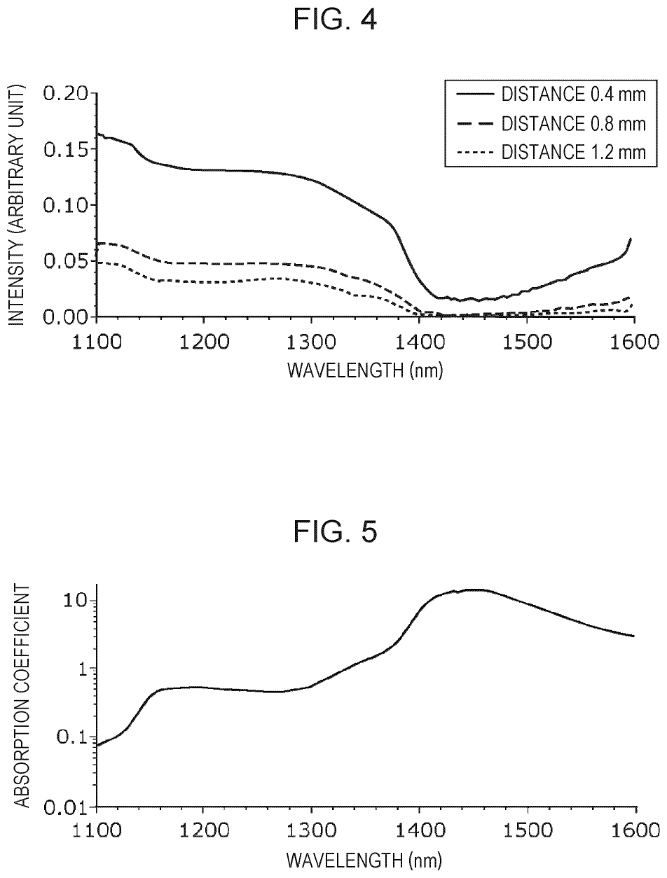

FIG. 4 is a diagram illustrating wavelength dependencies of intensities of the subsurface scattering light when the light is made incident on the skin while changing wavelengths thereof. To be more precise, FIG. 4 illustrates the wavelength dependencies of the intensities in the case where the light from an optical fiber core having a diameter of 400 μm and being pressed against the skin is caused to enter the skin, and the subsurface scattering light is received with another optical fiber core having a diameter of 400 μm and being pressed against the skin at center distances of 0.4 mm, 0.8 mm, and 1.2 mm away from the center of the optical fiber core used for causing the light to enter.

It is apparent from FIG. 4 that the subsurface scattering light at a wavelength greater than or equal to 1380 nm is attenuated significantly as compared with the subsurface scattering light at a wavelength less than 1380 nm.

Meanwhile, FIG. 5 is a diagram illustrating a wavelength dependency of an absorption coefficient of water. A high degree of correlation is found between the wavelength dependency of the absorption coefficient of water illustrated in FIG. 5 and the wavelength dependencies of the intensities of the subsurface scattering light illustrated in FIG. 4. Specifically, the attenuation of the subsurface scattering light is thought to be mainly due to the influence of resonance absorption by moisture contained in the skin.

As illustrated in FIG. 5, a value of the absorption coefficient of water at the wavelength greater than or equal to 1380 nm has a value that is greater than or equal to a value at the wavelength less than 1380 nm. In other words, the subsurface scattering light at the wavelength greater than or equal to 1380 nm is deemed to have a lower intensity than that of the subsurface scattering light at the wavelength less than 1380 nm. While the absorption coefficient of water illustrated in FIG. 5 is reduced at a wavelength greater than 1450 nm, the absorption coefficient of water reaches a minimum value at a wavelength in a range from about 1600 nm to 1700 nm, and has a higher value even at a wavelength greater than or equal to 1600 nm than the value at the wavelength of 1380 nm.

Meanwhile, the subsurface scattering light component is significantly influenced by the absorption by water inside the finger as described above. On the other hand, the surface-reflected light component does not enter the skin and is therefore influenced little by the absorption by the water. Accordingly, at the wavelength greater than or equal to 1380 nm where the subsurface scattering light component is significantly attenuated, the surface-reflected light component is mainly imaged out of the reflected light from the finger originating from the light projected onto the finger. As a consequence, the fingerprint image obtained by the imaging contains more information on the asperities on the surface that is useful for authentication. As described above, the inventor has found out that the imaging by use of the light at the wavelength greater than or equal to 1380 nm makes it possible to perform authentication at high accuracy or at a high speed while reducing the occurrence of authentication errors. This aspect applies not only to the case of obtaining the fingerprint image by shooting the image of the finger but also to a case of obtaining a palm print image by shooting an image of a palm.

Now, embodiments of the present disclosure conceived based on the above-mentioned knowledge will be described below.

An outline of an aspect of the present disclosure is as follows.

A contactless authentication system according to an aspect of the present disclosure includes: at least one illumination apparatus that projects illumination light onto a portion of a hand, the portion being not in contact of an object, the illumination light containing a light component in a wavelength range greater than or equal to 1380 nm; and an imaging apparatus that obtains at least one selected from the group consisting of a fingerprint image and a palm print image as authentication information by imaging the light component in the wavelength range in reflected light generated by reflection of the illumination light from the portion of the hand.

As described above, the imaging apparatus obtains the authentication information by imaging the reflected light that is reflected from the hand in the state of non-contact with the object. Here, the reflected light has the light component in the wavelength range greater than or equal to 1380 nm. Accordingly, it is possible to obtain the authentication information that contains a lot of information on asperities on a surface of the hand with less influence of the subsurface scattering light. An authentication error is less likely to occur as a consequence of carrying out the authentication by using the authentication information thus obtained. In this way, the contactless authentication system according to the present aspect can obtain the authentication information, which is capable of suppressing the occurrence of the authentication error, from the hand not in contact with the object.

For example, the authentication information may include information indicating a position of a sweat pore.

Accordingly, the authentication information includes the information indicating the position of the sweat pore, which is promising information for improving authentication accuracy. Thus, the occurrence of an authentication error can further be suppressed by using this authentication information for the authentication.

For example, the imaging apparatus may include a photoelectric conversion layer, and sensitivity of the photoelectric conversion layer may have a peak in the wavelength range greater than or equal to 1380 nm.

Accordingly, it is possible to increase the sensitivity of the imaging apparatus in the wavelength range greater than or equal to 1380 nm.

For example, the photoelectric conversion layer may include a quantum dot.

The quantum dot is likely to have a steep peak of light absorption. Accordingly, it is possible to realize the imaging apparatus that has high sensitivity to a specific wavelength greater than or equal to 1380 nm and has low sensitivity to a wavelength different from the specific wavelength.

For example, the photoelectric conversion layer may include a semiconducting carbon nanotube.

The semiconducting carbon nanotube is likely to have a steep peak of light absorption. Accordingly, it is possible to realize the imaging apparatus that has high sensitivity to a specific wavelength greater than or equal to 1380 nm and has low sensitivity to a wavelength different from the specific wavelength.

For example, the light component to be imaged by the imaging apparatus may contain a wavelength at which solar light is significantly attenuated on a ground surface. In other words, the imaging apparatus may obtain the authentication information within the wavelength range greater than or equal to 1380 nm by imaging the light component in a wavelength range including an attenuation wavelength of the solar light on the ground surface. Here, the attenuation wavelength of the solar light on the ground surface means such a wavelength that has a significant value of percentage of attenuation of the intensity of the solar light on the ground surface when the intensity of the solar light outside the atmosphere is compared with the intensity of the solar light on the ground surface.

Accordingly, it is possible to obtain the authentication information with relatively large influence of the reflected light while reducing the influence of the solar light. Hence, the occurrence of an authentication error is more suppressed by using the above-described authentication information for the authentication.

For example, the imaging apparatus may include an optical filter, and a transmittance of the optical filter with respect to light having a wavelength less than 1380 nm may be lower than a transmittance of the optical filter with respect to light having a wavelength greater than or equal to 1380 nm.

Accordingly, it is possible to increase the sensitivity of the imaging apparatus relatively in the wavelength range greater than or equal to 1380 nm.

For example, the at least one illumination apparatus may cyclically change an emission intensity of the illumination light, and the imaging apparatus may cyclically change sensitivity of the imaging apparatus in response to the change in the emission intensity of the illumination light.

Accordingly, it is possible to obtain as authentication information the images that are shot by changing a relation between a phase of the emission intensity of the illumination light and a phase of the sensitivity of the imaging apparatus. In other words, it is possible to obtain the image with large influence of the reflected light from the hand originating from the illumination light and the image with small influence thereof. Hence, it is possible to obtain the authentication information that can reduce influence of ambient light by acquiring a difference image between these images, for example.

For example, the at least one illumination apparatus may project the illumination light onto the hand in a first direction and in a second direction different from the first direction, and the imaging apparatus may image the reflected light originating from the illumination light projected onto the hand in the first direction and the reflected light originating from the illumination light projected onto the hand in the second direction.

As described above, a mode of forming shades on the asperities on the hand is changed by imaging the reflected light from the hand originating from the illumination light having the different directions of projection. Thus, it is possible to obtain images having different areas on the images with high contrast originating from shades of the asperities on the hand. As a consequence, it is possible to obtain the authentication information that contains a lot of information on the asperities in a wider range on the surface of the hand.

For example, the at least one illumination apparatus may include a first illumination apparatus that projects the illumination light onto the hand in the first direction, and a second illumination apparatus that projects the illumination light onto the hand in the second direction, and timing to project the illumination light from the first illumination apparatus onto the hand may be different from timing to project the illumination light from the second illumination apparatus onto the hand.

Accordingly, it is possible to project the illumination light onto the hand from directions of projection different from each other by adopting a simple structure.

For example, the at least one illumination apparatus may include an adjuster that changes the direction of projection of the illumination light onto the hand, and the at least one illumination apparatus may project the illumination light onto the hand in the first direction and the second direction by using the adjuster.

Accordingly, it is possible to project the illumination light onto the hand from directions of projection different from each other without increasing the number of the illumination apparatuses.

For example, the light component imaged by the imaging apparatus may be a light component in the reflected light in a wavelength range greater than or equal to 1380 nm and less than 2500 nm. In other words, the imaging apparatus may obtain at least one selected from the group consisting of the fingerprint image and the palm print image as the authentication information by imaging the light component in the reflected light of the illumination light, which has the wavelength range greater than or equal to 1380 nm and less than 2500 nm.

Accordingly, it is possible to obtain the clear authentication information with less thermal noise originating from the imaging apparatus and fewer components thermally radiated from a subject in the wavelength range less than 2500 nm, and so on.

An authentication method according to another aspect of the present disclosure includes: projecting illumination light onto a portion of a hand, the portion being not in contact of an object, the illumination light containing a light component in a wavelength range greater than or equal to 1380 nm; and obtaining at least one selected from the group consisting of a fingerprint image and a palm print image as authentication information by imaging the light component in the wavelength range in reflected light generated by reflection of the illumination light from the portion of the hand.

Accordingly, the method can obtain the authentication information that contains a lot of information on the asperities on the surface of the hand with less influence of the subsurface scattering light from the hand in the state of non-contact with the object as with the aforementioned contactless authentication system. Thus, the authentication method according to the present aspect can obtain the authentication information capable of suppressing the occurrence of the authentication error from the hand not in contact with the object.

Now, a description will be given of certain embodiments with reference to the drawings.

Note that each of the embodiments described below represents either a comprehensive or specific example. Numerical values, shapes, constituents, layouts and modes of connection of the constituents, steps, the orders of the steps, and the like depicted in the following embodiments are mere examples and are not intended to restrict the scope of the present disclosure. Meanwhile, of the constituents in the following embodiments, a constituent not defined in an independent claim will be described as an optional constituent. In the meantime, the respective drawings are not always illustrated precisely. Accordingly, scales and other factors do not always coincide with one another in the drawings, for example. It is to be also noted that the constituents which are substantially the same may be denoted by the same reference signs in the drawings, and overlapping explanations thereof may be omitted or simplified as appropriate.

In the present specification, terms that represent relations between elements, terms that represent shapes of the elements, and numerical ranges are not expressions that only represent precise meanings but are rather expressions that encompass virtually equivalent ranges with allowances of several percent, for example.

### Embodiment 1

### 1. Configuration of Contactless Authentication System

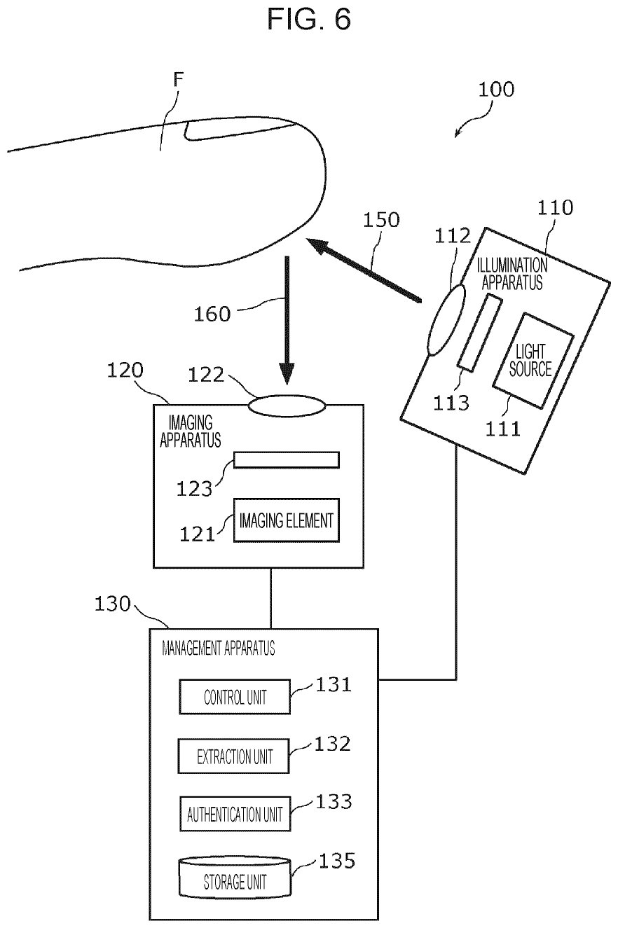

A configuration of a contactless authentication system according to the present embodiment will be described to begin with. FIG. 6 is a block diagram illustrating a schematic configuration of a contactless authentication system 100 according to the present embodiment.

As illustrated in FIG. 6, the contactless authentication system 100 includes an illumination apparatus 110, an imaging apparatus 120, and a management apparatus 130. The contactless authentication system 100 obtains authentication information from a hand not in contact with an object. To be more precise, the contactless authentication system 100 obtains the authentication information from at least part of the hand not in contact with the object. In an example illustrated in FIG. 6, the contactless authentication system 100 obtains the authentication information from a finger F being part of the hand of an authenticatee. The authentication information is any of a fingerprint image and a palm print image, or both the fingerprint image and the palm print image. In other words, the authentication information is an image obtained by imaging any of a finger and a palm, or both the finger and the palm. In the following, a description will be given of an example in which the contactless authentication system 100 obtains the authentication information, namely, a fingerprint image, from the finger F not in contact with the object.

In the contactless authentication system 100, the illumination apparatus 110 projects illumination light 150 onto the finger F, which is a subject not in contact with a glass surface and the like of a prism. Meanwhile, the imaging apparatus 120 obtains the fingerprint image as the authentication information by imaging reflected light 160 of the illumination light 150 that is reflected from the finger F. As mentioned above, the reflected light 160 includes surface-reflected light from the finger F and subsurface scattering light that is scatter-reflected light from the finger F. In the following, a description will be given of a case where the illumination apparatus 110 projects the illumination light 150 onto the finger F that is not in contact with any object. Nonetheless, the finger F may be partially in contact with an object. In this case, the illumination apparatus 110 projects the illumination light 150 at least onto a portion of the finger F not in contact with the object. The imaging apparatus 120 images the reflected light 160 of the illumination light 150 reflected from the portion of the finger F not in contact with the object.

The management apparatus 130, for example, controls operations of the illumination apparatus 110 and imaging apparatus 120, and performs a variety of information processing concerning the authentication information obtained by the imaging apparatus 120.

Now, details of respective constituents of the contactless authentication system 100 will be described below.

### 1.1. Illumination Apparatus

The illumination apparatus 110 includes a light source 111, an illumination optical system 112, and an optical filter 113.

The illumination apparatus 110 projects the illumination light 150, which has a light component in a wavelength range greater than or equal to 1380 nm, onto the finger F being the subject. The illumination light 150 has the light component in a wavelength greater than or equal to 1380 nm and less than 2500 nm, for example. In the present specification, light that does not contain visible light components will also be expressed as the “illumination light” for the sake of convenience.

The illumination light 150 may contain a light component at a wavelength less than 1380 nm. The illumination apparatus 110 projects the illumination light 150 having the light component in the wavelength range greater than or equal to 1380 nm as the main light component, for example. The aspect of the illumination light 150 having the light component in the wavelength range greater than or equal to 1380 nm as the main light component means that a value obtained by integrating products of emission intensities at the wavelength greater than or equal to 1380 nm and a quantum efficiency of an imaging element 121 is greater than or equal to 50% relative to a value obtained by integrating products of emission intensities and the quantum efficiency of the imaging element 121 throughout a wavelength range in which the imaging apparatus 120 to be described later in detail has sensitivity in an emission spectrum of the illumination light 150. Here, the wavelength range in which the imaging apparatus 120 has the sensitivity means a wavelength range in which the imaging apparatus 120 has the quantum efficiency that has the influence on an imaging result such as a wavelength range in which the imaging apparatus 120 has the quantum efficiency not equal to 0.

The wavelength range in which the imaging apparatus 120 has the sensitivity is determined mainly based on a photoelectric conversion material used for the imaging element 121 and on an optical filter 123. For example, in the case of the imaging element using an indium gallium arsenide compound as a typical photoelectric conversion material, the wavelength range in which the imaging apparatus has the sensitivity is roughly less than or equal to 1700 nm. In the case of the imaging element using a quantum dot containing lead sulfide as a core as the photoelectric conversion material, the wavelength range in which the imaging apparatus has the sensitivity is roughly less than or equal to 1600 nm although this range varies depending on a grain size and other factors of the quantum dot.

The illumination light 150 may have the light component in a wavelength range in which the imaging element 121 does not have sensitivity. The illumination light 150 may contain three types of light components including: (1) the light component in the wavelength range in which the imaging element 121 has the sensitivity, namely, the light component at the wavelength greater than or equal to 1380 nm; (2) the light component in the wavelength range in which the imaging element 121 has the sensitivity, namely, the light component at the wavelength less than 1380 nm; and (3) the light component in the wavelength range in which the imaging element 121 does not have the sensitivity. In the emission spectrum of the illumination light 150, a value obtained by integrating products of the emission intensities and the quantum efficiency of the imaging element 121 in the wavelength range in which the imaging element 121 has the sensitivity, namely, the wavelength range greater than or equal to 1380 nm is greater than or equal to a value obtained by integrating products of the emission intensities and the quantum efficiency of the imaging element 121 in the wavelength range in which the imaging element 121 has the sensitivity, namely, the wavelength range less than 1380 nm. A percentage of the light component (3) in the illumination light 150 is not limited to a particular value. Accordingly, when the imaging element 121 has the significant sensitivity only at the wavelength greater than or equal to 1380 nm, the illumination light 150 only needs to have the light component with a sufficient intensity for imaging at the wavelength greater than or equal to 1380 nm. For example, the illumination light 150 may contain the light components in a wide wavelength range from ultraviolet rays to far-infrared rays, such as light emitted from a xenon lamp.

The illumination apparatus 110 is disposed in such a way as to illuminate a region where the fingerprint of the finger F is present. Here, the finger F is not pressed against a glass surface and the like, or in other words, in a state of non-contact. The finger F to be irradiated with the illumination light 150 is not in contact with any object and is exposed in the air, for example. Moreover, the illumination apparatus 110 is disposed such that the reflected light 160 from the surface of the finger F originating from the illumination light 150 projected onto the finger F is made incident on the imaging apparatus 120.

Furthermore, the illumination apparatus 110 is disposed in such a way as to project the illumination light 150 at such an angle that fingerprint lines constituting projections in a fingerprint region shade grooves being located between the fingerprint lines and constituting recesses in the fingerprint region, for example. In other words, the illumination apparatus 110 is disposed in such a way as to project the illumination light 150 onto, for example, bottom portions of the grooves between the fingerprint lines in an oblique direction instead of a perpendicular direction.

In addition, a direction of projection of the illumination light 150 from the illumination apparatus 110 and an imaging direction by the imaging apparatus 120 are different from each other, for example. Nonetheless, the direction of projection of the illumination light 150 from the illumination apparatus 110 may be the same direction as the imaging direction by the imaging apparatus 120.

The light source 111 emits the light having the light component, or an emission intensity in other words, at the wavelength greater than or equal to 1380 nm. The light emitted from the light source 111 may contain the light component at the wavelength less than 1380 nm.

The light source 111 is the light source that emits the light in a wide wavelength range that encompasses, for example, both the light component at the wavelength greater than or equal to 1380 nm and the light component at the wavelength less than 1380 nm. Examples of this light source 111 include a halogen lamp, a xenon lamp, a supercontinuum light source, and the like.

Meanwhile, the light source 111 may be a light source that emits light having the light component that is concentrated on a specific wavelength range in the wavelength range greater than or equal to 1380 nm. The light source 111 emits light which has a central wavelength of its light component in the wavelength range greater than or equal to 1380 nm, and has a half width of the light component in the emission spectrum in a range equal to or less than several hundred nanometers, for example. Examples of this light source 111 include an LED, a laser diode, a superluminescent diode, and the like. To be more precise, the product M1450L3 manufactured by Thorlabs, Inc. of which emission spectrum is illustrated in FIG. 2, for example, has the central wavelength of about 1450 nm, and the half width of the light component thereof of about 100 nm. The product M1450L3 may be used as the light source 111. Alternatively, a laser diode having a central wavelength of its light component equal to 1550 nm and a half width of the light component less than or equal to 1 nm may be used as the light source 111, for example.

The illumination optical system 112 has a function to irradiate a subject with the light emitted from the light source 111. The illumination optical system 112 is disposed at a position where the light emitted from the light source 111 is made incident. For example, the illumination optical system 112 is formed from a lens, a mirror, and the like. When the light source 111 which emits light in a restricted direction, such as a shell-type light emitting diode is used, the illumination optical system 112 does not have to be provided to the illumination apparatus 110. Meanwhile, the illumination optical system 112 may include a shutter, a diaphragm, and the like as appropriate.

The optical filter 113 has a function to reduce the light component at the wavelength less than 1380 nm in the light emitted from the light source 111. The optical filter 113 is disposed on an optical path of the light emitted from the light source 111. The optical filter 113 is disposed between the light source 111 and the illumination optical system 112, for example. Here, the optical filter 113 may be disposed in such a way as to be located between the illumination optical system 112 and the finger F.

Examples of the optical filter 113 include an interference filter formed from a dielectric multi-layer film and an absorption filter formed from colored glass and other things. The optical filter 113 may be a long-pass filter that has a transmittance with respect to the light having the wavelength less than 1380 nm which is lower than a transmittance with respect to the light having the wavelength greater than or equal to 1380 nm, or may be a bandpass filter which has a wavelength range with a significantly high transmittance in a range around a specific central wavelength greater than or equal to 1380 nm. The wavelength range in which the bandpass filter has the significantly high transmittance may coincide with a wavelength at which the imaging apparatus 120 has especially high sensitivity. For example, the imaging element 121 of the imaging apparatus 120 has a peak of sensitivity in the wavelength range in which the bandpass filter has the significantly high transmittance. When the light source 111 emits the light having the light component at the wavelength greater than or equal to 1380 nm as the main light component, the optical filter 113 does not have to be provided to the illumination apparatus 110.

### 1.2. Imaging Apparatus

The imaging apparatus 120 includes the imaging element 121, an imaging optical system 122, and the optical filter 123. The imaging apparatus 120 has sensitivity at the wavelength greater than or equal to 1380 nm. For example, the imaging apparatus 120 includes the imaging element 121 having the sensitivity at the wavelength greater than or equal to 1380 nm, thereby imaging the light at the wavelength greater than or equal to 1380 nm.

The imaging apparatus 120 is disposed at a position of incidence of the reflected light 160 from the fingerprint lines being the projections of the finger F, the finger F being in the state of non-contact and irradiated with the illumination light 150.

The imaging apparatus 120 images the light component in the wavelength range greater than or equal to 1380 nm in the reflected light 160 from the region of the finger F where the fingerprint exists, the finger F being in the state of non-contact and irradiated with the illumination light 150. The imaging apparatus 120 may also image a light component in a wavelength range including an attenuation peak of solar light on a ground surface within the wavelength range greater than or equal to 1380 nm. Details of the wavelength range including the attenuation peak of the solar light will be described later. Meanwhile, the imaging apparatus 120 may also image a light component in the wavelength range greater than or equal to 1380 nm and less than 2500 nm in the reflected light 160.

Alternatively, the imaging apparatus 120 may image the reflected light 160 while defining the wavelength range greater than or equal to 1380 nm as a main imaging component. The imaging apparatus 120 images the reflected light 160 while defining the wavelength range greater than or equal to 1380 nm and less than 2500 nm as the main imaging component, for example. The main imaging component has the meaning to be described below.

The imaging element 121 has a function to generate signal charges by using incidence of photons. The imaging apparatus 120 images the reflected light 160 by using the imaging element 121. The imaging element 121 generates the signal charges being the imaging component by using the incidence of the light at the wavelength greater than or equal to 1380 nm. In other words, the imaging element 121 has the sensitivity to the wavelength greater than or equal to 1380 nm. In this instance, a ratio of the signal charges to be generated by each photon is called a quantum efficiency. The quantum efficiency has a wavelength dependency. Meanwhile, an amount of photons incident on the imaging element 121 (that is, the light component of the reflected light 160) also has a wavelength dependency. For this reason, an amount of signal charges to be generated by light at a certain wavelength satisfies the following formula 1:

(amount of signal charges generated by light at certain wavelength)=(amount of photons at certain wavelength)×(quantum efficiency at certain wavelength) formula 1

Here, a total amount of signal charges generated by the incidence of the reflected light 160 to the imaging element 121 is equivalent to a value obtained by integrating the formula 1 throughout the entire wavelength range in terms of the reflected light 160. The entire wavelength range means the entire range of the wavelength of the light targeted for imaging, which is the entire wavelength range of the imaging element 121 having the quantum efficiency not equal to 0, for example.

The light at a wavelength having a large value derived from the formula 1 generates more signal charges than the light at a wavelength having a small value derived from the formula 1 does. In other words, the light having the large value brings about a larger influence on the imaging result. The wavelength range for defining the main imaging component means the wavelength range in which the signal charges are mainly generated. To image the reflected light 160 while defining the wavelength range greater than or equal to 1380 nm as the main imaging component may represent, for example, an aspect in which the amount of signal charges generated by the reflected light 160 in the wavelength range greater than or equal to 1380 nm is greater than or equal to 50%, or greater than or equal to 90% relative to the total amount of signal charges generated by the reflected light 160.

As described above, the imaging apparatus 120 images the reflected light 160 while defining the wavelength range greater than or equal to 1380 nm as the main imaging component. As apparent from the formula 1, the illumination light 150 needs to have the light component at the wavelength greater than or equal to 1380 nm while the imaging element 121 needs to have the quantum efficiency not equal to 0 at the wavelength greater than or equal to 1380 nm in order to define the wavelength range greater than or equal to 1380 nm as the main imaging component. For example, the quantum efficiency of the imaging element 121 for the light having the wavelength greater than or equal to 1380 nm is higher than the quantum efficiency of the imaging element 121 for the light having the wavelength less than 1380 nm. This aspect means that a value obtained by integrating the quantum efficiencies at the wavelength greater than or equal to 1380 nm is greater than a value obtained by integrating the quantum efficiencies at the wavelength less than 1380 nm in light of the wavelength dependency of the quantum efficiency of the imaging element 121. Meanwhile, a value obtained by integrating the quantum efficiencies at the wavelength greater than or equal to 1380 nm and less than 2500 nm may be greater than a value obtained by integrating the quantum efficiencies at the wavelength greater than or equal to 380 nm and less than 1380 nm in light of the wavelength dependency of the quantum efficiency of the imaging element 121. Alternatively, a wavelength at which the imaging element 121 has high sensitivity, that is, a high quantum efficiency may be brought in line with a wavelength at which the illumination light 150 has a large light component.

The wavelength range in which the imaging apparatus 120 performs imaging as the main imaging component is a range of a near-infrared region less than 2500 nm, for example. A middle-infrared region at a wavelength greater than or equal to 2500 nm and a far-infrared region at a wavelength greater than or equal to 4000 nm bring about a lot of thermal noise to the imaging element 121 and the subject per se brings about more thermal radiation components. For this reason, it may be difficult to obtain clear authentication information when imaging is performed in the middle-infrared region or the far-infrared region.

For example, the imaging element 121 includes a photoelectric conversion material that converts photons into electric charges, a peripheral circuit for reading the electric charges generated by the photoelectric conversion material as the signal charges, and the like. Examples of the photoelectric conversion material for allowing the imaging element 121 to have the sensitivity to the wavelength greater than or equal to 1380 nm include an indium gallium arsenide compound, a quantum dot containing lead sulfide or lead selenide as a core, a semiconducting carbon nanotube, and the like.

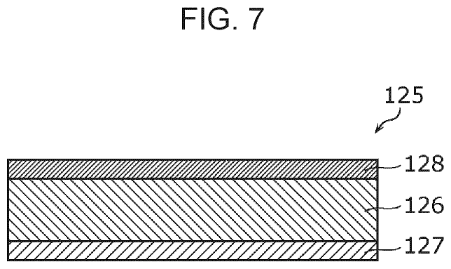

The imaging element 121 is a laminated image sensor provided with a photoelectric conversion element including a photoelectric conversion layer containing the photoelectric conversion material. FIG. 7 is a sectional view illustrating an example of a schematic configuration of a photoelectric conversion element 125 provided to the imaging element 121. As illustrated in FIG. 7, the photoelectric conversion element 125 includes a pixel electrode 127, a counter electrode 128 disposed opposite to the pixel electrode 127, and a photoelectric conversion layer 126 located between the pixel electrode 127 and the counter electrode 128.

The photoelectric conversion layer 126 contains the photoelectric conversion material that absorbs the incident light and generates hole-electron pairs as the signal charges. The photoelectric conversion material is a semiconducting inorganic material or a semiconducting organic material, which absorbs the light at the wavelength greater than or equal to 1380 nm, for example. The photoelectric conversion layer 126 includes either the quantum dot or the semiconducting carbon nanotube, or both the quantum dot and the semiconducting carbon nanotube as the photoelectric conversion material, for example.

Each of the semiconductor quantum dot and the semiconducting carbon nanotube has a steep peak of light absorption. A light absorption peak wavelength of the quantum dot can be controlled by the material and grain sizes of the semiconductor quantum dot. A light absorption peak wavelength of the semiconducting carbon nanotube can be controlled by chirality of the semiconducting carbon nanotube. Accordingly, the wavelength in which the imaging element has the sensitivity can be easily adjusted by using at least one selected from the group consisting of the semiconductor quantum dot and the semiconducting carbon nanotube as the photoelectric conversion material. Thus, it is possible to realize the imaging element 121 that has high sensitivity to a specific wavelength and has low sensitivity to a wavelength different from the specific wavelength. For example, when the photoelectric conversion layer 126 includes at least one selected from the group consisting of the quantum dot and the semiconducting carbon nanotube each having a light absorption peak at the wavelength greater than or equal to 1380 nm, it is possible to realize the imaging element 121 having high sensitivity to the wavelength greater than or equal to 1380 nm and low sensitivity to the wavelength less than 1380 nm.

The pixel electrode 127 is an electrode for collecting the signal charges generated by the photoelectric conversion layer 126. The peripheral circuit of the imaging element 121 reads out the signal charges collected by the pixel electrode 127. The pixel electrode 127 is formed by using a conductive material. Examples of the conductive material include a metal such as aluminum and copper, a metal nitride, and polycrystalline silicon provided with conductivity by being doped with an impurity.

The counter electrode 128 is a transparent electrode formed from a transparent conductive material, for example. The counter electrode 128 is disposed on a light incident side of the photoelectric conversion layer 126. Accordingly, the light that passes through the counter electrode 128 is made incident on the photoelectric conversion layer 126. In the present specification, the term “transparent” means an aspect of passage of at least part of the light at the wavelength range targeted for detection.

A voltage is applied to the counter electrode 128. An electric potential difference between the counter electrode 128 and the pixel electrode 127 can be set to and maintained at a desired electric potential difference by adjusting the voltage to be applied to the counter electrode 128. The counter electrode 128 is formed by using a transparent conducting oxide (TCO) such as ITO, IZO, AZO, FTO, SnO₂, TiO₂, and ZnO.

As described above, in the laminated image sensor, the electric potential of the counter electrode 128 relative to the electric potential of the pixel electrode 127 is controlled. Accordingly, the pixel electrode 127 can collect any of the holes and the electrons as the signal charges out of the hole-electron pairs generated in the photoelectric conversion layer 126 as a consequence of the photoelectric conversion.

The imaging element 121 includes multiple pixels each of which reads out the signal charges, and each pixel is provided with the photoelectric conversion element 125. In this case, the pixel electrodes 127 are provided one by one to the respective pixels. In the meantime, the photoelectric conversion layer 126 and the counter electrode 128 may be provided across two or more pixels.

Here, the photoelectric conversion element 125 may be provided with other layers including a charge transport layer, a charge blocking layer, a buffer layer, and the like which are located between the photoelectric conversion layer 126 and the pixel electrode 127, between the photoelectric conversion layer 126 and the counter electrode 128, or both between the photoelectric conversion layer 126 and the pixel electrode 127 and between the photoelectric conversion layer 126 and the counter electrode 128.

Referring to FIG. 6 again, the imaging optical system 122 has a function to form the image of the subject on the imaging element 121. The imaging optical system 122 is disposed on the incident side of the reflected light 160 on the imaging element 121. The imaging optical system 122 causes the reflected light 160 incident on the imaging optical system 122 to enter the imaging element 121. For example, the imaging optical system 122 is formed from a lens, a curved surface mirror, and the like. Components that have fine transmittances and imaging performances in the wavelength range to be subjected to imaging as the main imaging component are selected as the imaging optical system 122, for example.

The optical filter 123 transmits the light component at the wavelength greater than or equal to 1380 nm and blocks or attenuates the light component at the wavelength less than 1380 nm, for example. In other words, the optical filter 123 has a function to reduce the light component at the wavelength less than 1380 nm from the reflected light 160. The optical filter 123 is disposed between the imaging optical system 122 and the imaging element 121 or on the incident side of the reflected light 160 in the imaging optical system 122.

For instance, the optical filter 123 is a long-pass filter that has a transmittance with respect to the light having the wavelength less than 1380 nm which is lower than a transmittance with respect to the light having the wavelength greater than or equal to 1380 nm. Examples of the optical filter 123 include an interference filter formed from a dielectric multi-layer film, and an absorption filter formed from colored glass and the like.

Alternatively, the optical filter 123 may be a bandpass filter which has a wavelength range with a high transmittance only in a range around a specific central wavelength greater than or equal to 1380 nm. The specific central wavelength for the bandpass filter may substantially coincide with the wavelength at which the illumination light 150 has the large light component. For instance, a peak wavelength of the light component of the illumination light 150 may be included in the range around the specific central wavelength of the bandpass filter. Meanwhile, when the optical filter 113 of the illumination apparatus 110 is the bandpass filter, the specific central wavelength of the bandpass filter in the optical filter 113 may be equal to that of the bandpass filter in the optical filter 123. When, for example, the imaging element 121 has the high sensitivity only to the wavelength greater than or equal to 1380 nm, the optical filter 123 need not be provided to the imaging apparatus 120.

As described above, it is possible to reduce the light component at the wavelength less than 1380 nm to reach the imaging element 121 by providing the imaging apparatus 120 with the optical filter 123. As a consequence, it is possible to reduce a ratio of incidence of the light at the wavelength less than 1380 nm on the imaging element 121 in a situation where there is a large amount of light other than the reflected light 160 from the finger F originating from the illumination light 150 emitted by the illumination apparatus 110 such as solar light and ambient illumination light in an outdoor environment.

Here, the imaging element 121 may include multiple pixels each of which reads out the signal charges, and only certain pixels thereof may perform imaging while defining the wavelength range greater than or equal to 1380 nm as the main imaging component. For example, the imaging element 121 may include four types of pixels, namely, a red (R) pixel, a green (G) pixel, a blue (B) pixel, and infrared (IR) pixel, and may perform imaging while defining the wavelength range greater than or equal to 1380 nm as the main imaging component by using information based on the signal charges read out with the IR pixel only. Meanwhile, information based on the signal charges read out with the R pixel, the G pixel, and the B pixel which image visible light may be used for checking the presence of the subject to be authenticated. In the meantime, a determination may be made as to whether the subject is a true finger of a living body or a false finger by comparing an imaging result by using the IR pixel with an imaging result by using the rest of the pixels. Details of a method of determining a false finger will be discussed later in the chapter of other embodiments.

### 1.3. Wavelength Range in Imaging

In the contactless authentication system 100 according to the present embodiment, the imaging apparatus 120 performs imaging while defining the wavelength range greater than or equal to 1380 nm as the main imaging component. In the contactless authentication system 100, the light source 111 of the illumination apparatus 110, the imaging element 121 of the imaging apparatus 120, and other components are selected so as to perform the imaging while defining the above-mentioned wavelength range as the main imaging component. Meanwhile, in the contactless authentication system 100, the optical filter 113 that restricts the wavelength range of the illumination light 150 and the optical filter 123 that restricts an imaging wavelength range may be selected so as to perform the imaging while defining the above-mentioned wavelength range as the main imaging component.

Alternatively, the imaging apparatus 120 may perform the imaging while defining a specific wavelength range as the main imaging component. Here, the specific wavelength range falls within the wavelength range greater than or equal to 1380 nm to be originally defined as the main imaging component. The specific wavelength range will be selected from the following viewpoints, for instance.

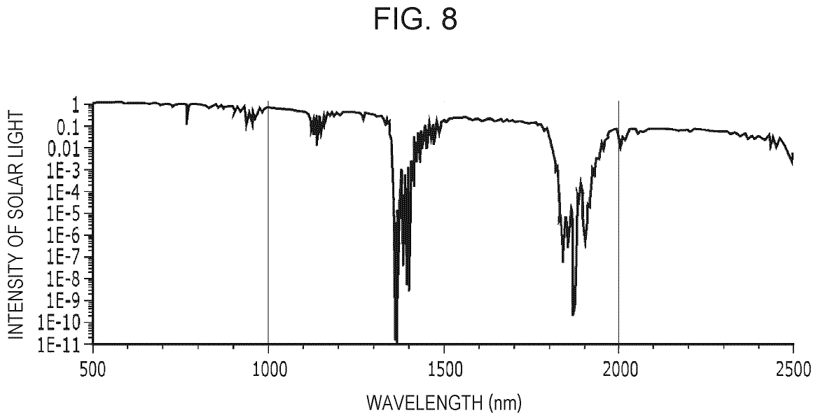

A first viewpoint is an intensity of solar light. FIG. 8 is a diagram illustrating a wavelength dependency of an intensity of solar light on a ground surface. As illustrated in FIG. 8, the intensity of the solar light reaching the ground surface exhibits significant variations depending on the wavelength. Specifically, in the wavelength range greater than or equal to 1380 nm, the intensity of the solar light reaching the ground surface exhibits significant attenuation in a wavelength range from 1380 nm to 1500 nm and a wavelength range from 1780 nm to 1990 nm. This attenuation is attributed to absorption of the solar light by the atmosphere. It is possible to reduce a ratio of incidence of the solar light on the imaging element 121 by using the wavelength at which the solar light is attenuated as mentioned above. The imaging apparatus 120 performs imaging by defining a wavelength range including the wavelength at which the solar light is attenuated on the ground surface as the main imaging component. As a consequence, the imaging by the imaging apparatus 120 is more likely to be carried out by using the reflected light 160. In the meantime, since the attenuation of the solar light is largely affected by the absorption by the moisture in the atmosphere, the subsurface scattering light also tends to be reduced by the influence of absorption by the moisture in the skin at the wavelength at which the solar light intensity is reduced. Accordingly, influence of ambient light and the subsurface scattering light is reduced so that the imaging can be carried out in a more intended way, and contrast of the fingerprint image can be improved as a consequence.

The influence of the solar light can be adjusted by the optical filter 123 provided to the imaging apparatus 120, for example. The influence of the solar light can be adjusted by using a central wavelength and a half width of a transmission band of the bandpass filter when the optical filter 123 is the bandpass filter, for instance.

When using a bandpass filter having the half width of the transmission band equal to about 10 nm, it is possible to reduce the intensity of the solar light that passes through the bandpass filter to about one-tenth or less relative to the relevant intensity in the case where the central wavelength of the bandpass filter is set to a visible range by setting the central wavelength of the transmission band to a wavelength range from 1380 nm to 1420 nm or to a wavelength range from 1820 nm to 1940 nm.

Likewise, when using a bandpass filter having the half width of the transmission band equal to about 50 nm, it is possible to reduce the intensity of the solar light that passes through the bandpass filter to about one-tenth or less relative to the relevant intensity in the case where the central wavelength of the bandpass filter is set to a visible range by setting the central wavelength of the transmission band to a wavelength range from 1380 nm to 1430 nm.

Meanwhile, in order to define the wavelength range including the wavelength of the attenuation peak of the solar light mentioned above as the main imaging component of the imaging apparatus 120, the light source 111 of the illumination apparatus 110 may adopt any of a light emitting diode, a laser diode, or a superluminescent diode having an emission peak within this wavelength range. In the meantime, when the optical filter 123 is the above-described bandpass filter, the light source 111 of the illumination apparatus 110 may have an emission peak within the transmission band of the bandpass filter.

A second viewpoint is eye safety. When the light source 111 is the laser diode, there is a limitation of available emission intensity in light of safety. Such an allowable intensity in light of safety depends on the wavelength. For example, a laser beam in a wavelength range from 1400 nm to 2600 nm is mostly absorbed by the eyeball and has less influence on the retina. Accordingly, the allowable intensity of this laser beam is higher than that of a laser beam at a wavelength outside the aforementioned wavelength range. The imaging apparatus 120 can obtain an image with less noise in a shorter time by using the light source 111 having higher output. Hence, the imaging apparatus 120 performs the imaging while defining the wavelength range of the laser beam emitted from the laser diode used as the light source 111 as the main imaging component, for example. A laser diode that emits a laser beam having a wavelength of 1550 nm, for instance, is eye-safe and a high-output product of such a laser diode is easily available.

A third viewpoint is the sensitivity of the imaging element 121. The imaging element 121 having the high sensitivity to the specific wavelength and having the low sensitivity to the wavelength different from the specific wavelength can be realized by adopting the quantum dot or the semiconducting carbon nanotube as the photoelectric conversion material used in the imaging element 121 as described above. Accordingly, the imaging apparatus 120 performs imaging while defining the wavelength range of light absorption originating from the light absorption peak of the photoelectric conversion material as the main imaging component, for example. The semiconducting carbon nanotube, for instance, has a characteristic of a resonant wavelength being a steep light absorption peak wavelength, which varies with a physical quantity called the chirality. The resonance of the semiconducting carbon nanotube having the single chirality has a narrow half width of several tens of nanometers to about a hundred nanometers. Accordingly, the imaging element 121 having the specifically high sensitivity to the wavelength range of light absorption originating from the resonant wavelength can be realized by using the semiconducting carbon nanotube as the photoelectric conversion material.

For example, the semiconducting carbon nanotube with the chirality (9, 8) has the resonant wavelength of about 1450 nm, while the semiconducting carbon nanotube with the chirality (10, 6) has the resonant wavelength of about 1400 nm. By using the above-mentioned semiconducting carbon nanotube having the resonant wavelength greater than or equal to 1380 nm as the photoelectric conversion material and bringing the peak of the wavelength of the light emitted from the light source 111 in line with the resonant wavelength, it is possible to reduce the influence of the ambient light having the wavelength other than the neighborhood of the resonant wavelength.

A detailed description is found in Japanese Patent No. 6778876 filed by the inventor of the present specification regarding details of the imaging element using the semiconducting carbon nanotube as the photoelectric conversion material.

### 1.4. Configurations of Management Apparatus and Others

The management apparatus 130 is a computer provided with a control unit 131, an extraction unit 132, an authentication unit 133, and a storage unit 135, for example.

The control unit 131 is a processing unit for controlling operations of the illumination apparatus 110 and the imaging apparatus 120. The control unit 131 outputs various control signals and the like to the illumination apparatus 110 and the imaging apparatus 120.

The extraction unit 132 is a processing unit for extracting characteristic information from authentication information being an imaging result (namely, the fingerprint image and the like).

The authentication unit 133 is a processing unit for carrying out determination, individual authentication, and the like by, for example, comparing the information extracted by the extraction unit 132 with information registered in the past, such as information registered with the storage unit 135, and comparing images shot by the imaging apparatus 120.

The processing units including the control unit 131, the extraction unit 132, the authentication unit 133, and the like may be implemented by, for example, one or more processors, or implemented by any of a microcomputer, a dedicated circuit, and the like.

The storage unit 135 is a storage device for storing the imaging result and the information to be used for the processing by the processing units. Moreover, the storage unit 135 stores programs to be executed by the processing units including the control unit 131, the extraction unit 132, the authentication unit 133, and the like. For example, the storage unit 135 is realized by any of a semiconductor memory, a hard disk drive (HDD), and the like.

Note that the respective constituents of the management apparatus 130 may be separately provided to two or more apparatuses, or at least one of the constituents of the management apparatus 130 may be provided to the illumination apparatus 110 or the imaging apparatus 120.

The contactless authentication system 100 may further include a sensor such as a human sensor for detecting a hand. Alternatively, the contactless authentication system 100 may use the imaging apparatus 120 as a sensor. For example, the control unit 131 may obtain a result of detection by the sensor, and start projection of the illumination light 150 from the illumination apparatus 110 and imaging with the imaging apparatus 120 by using the detection of the finger F by the sensor as a trigger.

### 2. Operation Examples of Contactless Authentication System

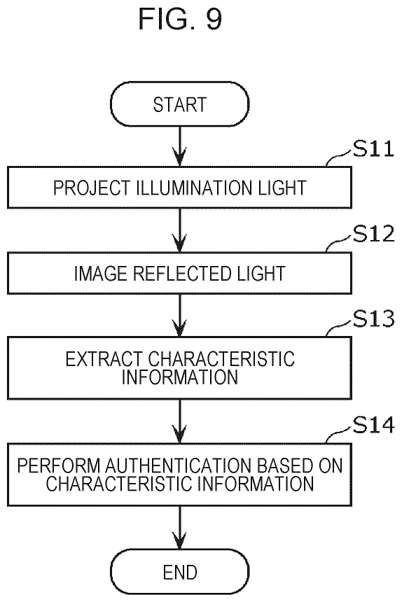

Next, operations of the contactless authentication system 100 according to the present embodiment will be described. To be more precise, a description will be given of an authentication method to be carried out by the contactless authentication system 100 designed to obtain the authentication information from the hand not in contact with an object. FIG. 9 is a flowchart illustrating an operation example of the contactless authentication system 100 according to the present embodiment.

As illustrated in FIG. 9, the illumination apparatus 110 projects the illumination light 150 having the light component at the wavelength greater than or equal to 1380 nm onto the finger F to begin with (step S11). The illumination apparatus 110 projects the illumination light 150 based on control by the control unit 131 or an operation of a user, for example. Here, the illumination apparatus 110 may constantly project the illumination light 150 throughout the operation of the contactless authentication system 100.

Next, the imaging apparatus 120 images the reflected light 160 generated by reflection of the illumination light 150 from the finger F after being projected onto the finger F while defining the wavelength range greater than or equal to 1380 nm as the main imaging component (step S12). The imaging apparatus 120 images the reflected light 160 based on control by the control unit 131 or an operation of the user, for example. Accordingly, the imaging apparatus 120 obtains the fingerprint image being the imaging result as the authentication information. Here, the fingerprint image may include information indicating positions of the sweat pores on the finger F as described with reference to the images illustrated in FIG. 1. The imaging apparatus 120 outputs the obtained fingerprint image to the management apparatus 130, for example.

Next, the extraction unit 132 of the management apparatus 130 obtains the fingerprint image from the imaging apparatus 120, and extracts the characteristic information being the information indicating characteristics of the finger F used for authentication (step S13). The extraction unit 132 extracts at least one of pieces of information on a pattern of the fingerprint, distribution of branching points and the like in the fingerprint, distribution of the sweat pores, or the like as the characteristic information.

Next, the authentication unit 133 performs authentication based on the characteristic information extracted by the extraction unit 132 (step S14). For example, the storage unit 135 stores information that indicates authentication candidates and pieces of their characteristic information in an associated manner, and the authentication unit 133 performs individual authentication by checking the characteristic information extracted by the extraction unit 132 against the characteristic information stored in the storage unit 135. The authentication unit 133 outputs information for notifying the authenticatee of an authentication result, for example. The extraction of the characteristic information, the check of the characteristic information, and other operations in steps S13 and S14 may apply publicly known fingerprint authentication techniques.

Here, the processing in steps S13 and S14 may be carried out by an external apparatus.

As described above, in the contactless authentication system 100, the imaging apparatus 120 images the reflected light 160 from the finger F in the state of non-contact with the object while defining the wavelength range greater than or equal to 1380 nm as the main imaging component, thereby obtaining the fingerprint image as the authentication information. Accordingly, it is possible to obtain the authentication information containing a lot of information on the asperities of the fingerprint on the finger F with less influence of the subsurface scattering light. For example, the fingerprint image is shot at high contrast by the imaging apparatus 120. Since the authentication unit 133 performs authentication by using the fingerprint image thus obtained, an authentication error is less likely to occur. As described above, the contactless authentication system 100 can obtain the authentication information capable of suppressing the occurrence of an authentication error from the finger F not in contact with the object.

### Embodiment 2

Next, a contactless authentication system according to Embodiment 2 will be described. Embodiment 2 will describe an example of a contactless authentication system including multiple illumination apparatuses. In the following description of Embodiment 2, a description will be given mainly on features different from those of Embodiment 1, and explanations of common features will be simplified or omitted.

### 1. Configuration of Contactless Authentication System

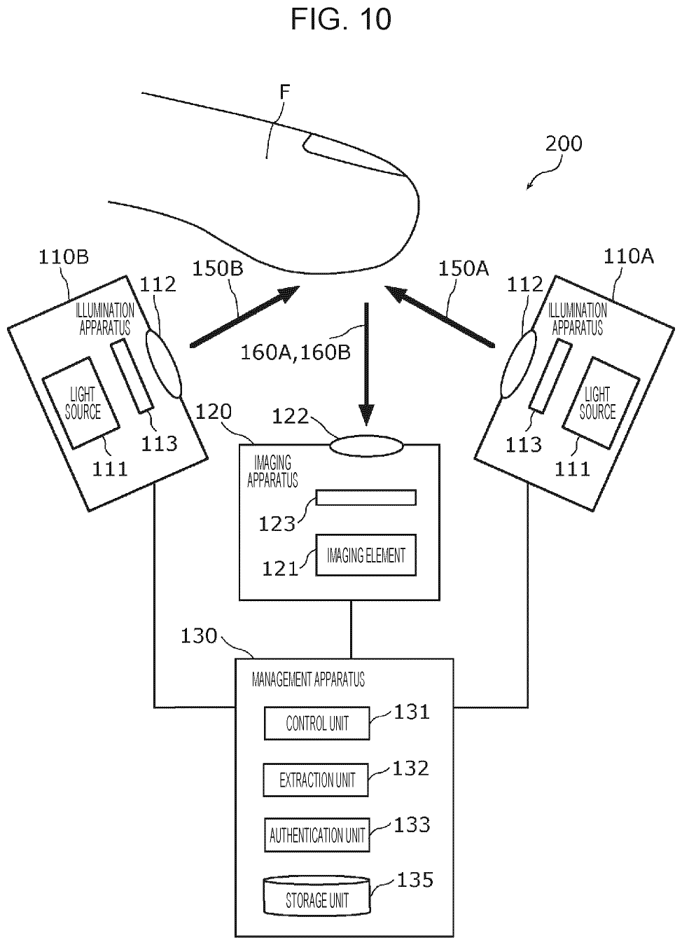

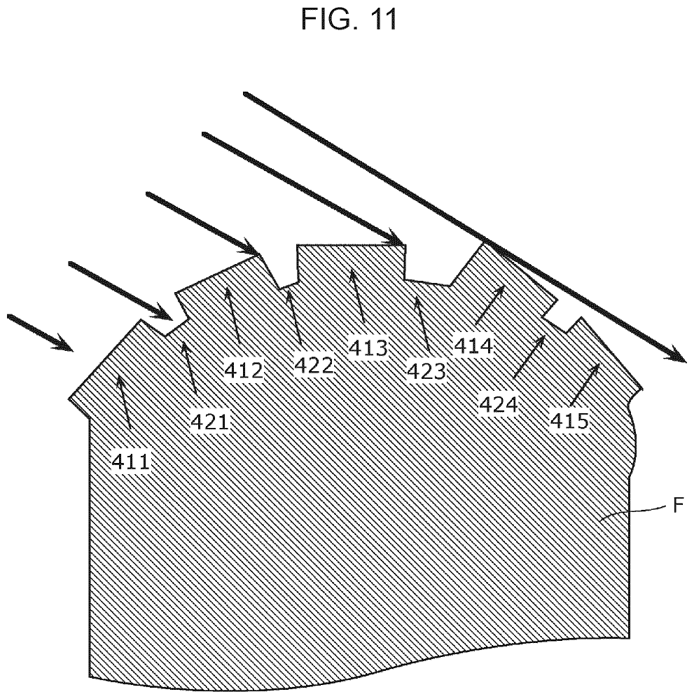

FIG. 10 is a block diagram illustrating a schematic configuration of a contactless authentication system 200 according to the present embodiment. As illustrated in FIG. 10, in comparison with the contactless authentication system 100 according to Embodiment 1, the contactless authentication system 200 is different in that an illumination apparatus 110A and an illumination apparatus 110B are provided as multiple illumination apparatuses instead of the single illumination apparatus 110. In other words, the contactless authentication system 200 according to Embodiment 2 includes the illumination apparatus 110A and the illumination apparatus 110B as the multiple illumination apparatuses, the imaging apparatus 120, and the management apparatus 130.

Each of the illumination apparatus 110A and the illumination apparatus 110B includes the light source 111, the illumination optical system 112, and the optical filter 113 as with the illumination apparatus 110. The illumination apparatus 110A irradiates the finger F with illumination light 150A while the illumination apparatus 110B irradiates the finger F with illumination light 150B having a direction of projection different from that of the illumination light 150A. The illumination apparatus 110A and the illumination apparatus 110B irradiate the finger F with the illumination light 150A and the illumination light 150B in directions different from each other. Here, the number of the illumination apparatuses provided to the contactless authentication system 200 is two apparatuses in the example illustrated in FIG. 10. However, three or more apparatuses may be provided instead. Meanwhile, the illumination apparatus 110A and the illumination apparatus 110B may be the apparatuses that are incorporated into a shared casing and the like.

In the contactless authentication system 200, the imaging apparatus 120 images reflected light 160A from the finger F originating from the illumination light 150A and reflected light 160B from the finger F originating from the illumination light 150B.

According to the above-described configuration, the contactless authentication system 200 of the present embodiment projects the illumination light 150A and the illumination light 150B in multiple directions of projection unlike Embodiment 1 in which the illumination light 150 is projected in one direction of projection. Meanwhile, in the contactless authentication system 200, the illumination apparatuses to project the illumination light can be switched sequentially, and the illumination apparatus 110A and the illumination apparatus 110B irradiate the finger F with the illumination light 150A and the illumination light 150B, respectively, at different timings. In the contactless authentication system 200, the illumination apparatus 110A and the illumination apparatus 110B irradiate the finger F with the illumination light 150A and the illumination light 150B, respectively, at different timings based on control by the control unit 131 or an operation of the user, for example.

The following advantages are brought about as a consequence of causing the contactless authentication system 200 to sequentially switch and project the illumination light 150A and the illumination light 150B in the directions of projection different from each other.

As described above with reference to FIG. 3, the image of the fingerprint is clearly shot with improved contrast in the case where the illumination light is projected onto the projections on the finger surface while the recesses on the finger surface are shaded.

FIG. 11 is a conceptual diagram of irradiation of the finger surface with the illumination light. In FIG. 11, the illumination light projected in an oblique direction relative to a direction of extension (a vertical direction in FIG. 11) of the finger F is indicated with arrows. As illustrated in FIG. 11, the finger F in the state of non-contact with any object forms a three-dimensionally curved surface. Here, when the illumination light has the direction of projection as illustrated in FIG. 11, a first projection 411, a second projection 412, and a third projection 413 on the finger F are exposed to a lot of the illumination light. On the other hand, a fourth projection 414 and a fifth projection 415 on the finger F are barely exposed to the illumination light.

Meanwhile, a first recess 421 on the finger F is exposed to the illumination light because nothing blocks the light. In the meantime, on the finger F, the second projection 412 shades a second recess 422 against the illumination light, and the third projection 413 shades a third recess 423 against the illumination light. Accordingly, the second recess 422 and the third recess 423 are not exposed to the illumination light. On the other hand, a fourth recess 424 as well as the surrounding projections are not exposed to the illumination light.

A region on the finger F where the image of the finger F is shot most clearly in order to increase contrast of the fingerprint image is each recess not exposed to the illumination light, which is sandwiched between the projections exposed to the illumination light. In the situation illustrated in FIG. 11, the image in the vicinity of the second recess 422 is shot most clearly.

As described above, the contrast of the fingerprint image depends on the direction of projection of the illumination light relative to the three-dimensional shape of the finger F and the three-dimensional shape of the fingerprint. Accordingly, an illuminated portion on the finger F and a position of a portion on the finger F where the recess is shaded can be changed by changing the direction of projection of the illumination light, thereby changing a high-contrast region in the fingerprint image. Thus, it is possible to obtain the high-contrast fingerprint image across a wide range on the finger F by sequentially changing the direction of projection of the illumination light. While FIG. 10 illustrates the example of providing the two illumination apparatuses, it is obvious that the range on the finger F which can be imaged at higher contrast becomes even wider if the direction of projection of the illumination light can be changed more often by providing a larger number of the illumination apparatuses.

Meanwhile, the change in contrast of the fingerprint due to the change in the direction of projection of the illumination light is caused by the three-dimensionality of the finger F and the fingerprint. Accordingly, such a change in contrast does not occur in the case of a false fingerprint image printed on a flat paper sheet or a false fingerprint image displayed on a liquid crystal display device and the like. Therefore, it is also possible to use information on the change in contrast of the fingerprint image that varies in accordance with the change in the direction of projection of the illumination light for the purpose of determination as to whether or not the fingerprint is true or false in order to suppress fraudulent authentication by using the false fingerprint.

### 2. Operation Examples of Contactless Authentication System

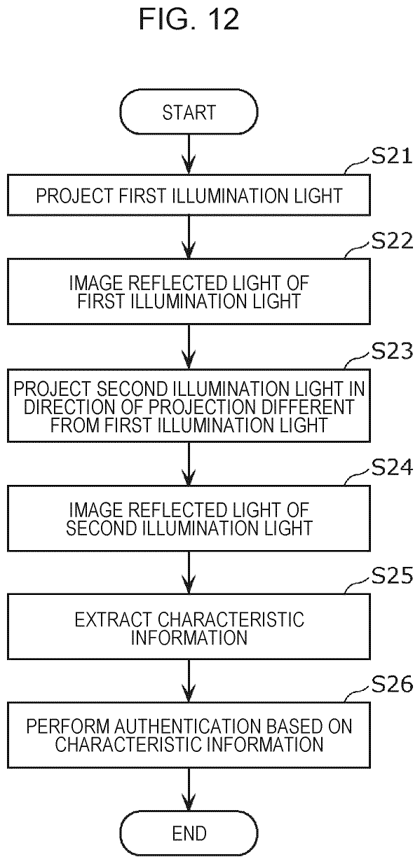

Next, operations of the contactless authentication system 200 according to the present embodiment will be described. FIG. 12 is a flowchart illustrating an operation example of the contactless authentication system 200 according to the present embodiment.

As illustrated in FIG. 12, the illumination apparatus 110A being a first illumination apparatus projects the illumination light 150A being first illumination light onto the finger F to begin with (step S21). Then, the imaging apparatus 120 images the reflected light 160A generated by reflection of the illumination light 150A from the finger F after being projected onto the finger F (step S22). Accordingly, the imaging apparatus 120 obtains a first fingerprint image being the imaging result as the authentication information. The imaging apparatus 120 outputs the obtained first fingerprint image to the management apparatus 130, for example. The extraction unit 132 of the management apparatus 130 obtains the first fingerprint image from the imaging apparatus 120, and stores the image in the storage unit 135.

Next, the illumination apparatus 110B being a second illumination apparatus projects the illumination light 150B being second illumination light having a direction of projection different from that of the first illumination light onto the finger F (step S23). In this instance, the illumination apparatus 110A is turned off and does not project the illumination light 150A onto the finger F. Then, the imaging apparatus 120 images the reflected light 160B generated by reflection of the illumination light 150B from the finger F after being projected onto the finger F (step S24). Accordingly, the imaging apparatus 120 obtains a second fingerprint image being the imaging result as the authentication information. The imaging apparatus 120 outputs the obtained second fingerprint image to the management apparatus 130, for example. The extraction unit 132 of the management apparatus 130 obtains the second fingerprint image from the imaging apparatus 120, and stores the image in the storage unit 135.

Next, the extraction unit 132 extracts the characteristic information from the first fingerprint image and the second fingerprint image stored in the storage unit 135 (step S25). The extraction unit 132 compares the first fingerprint image with the second fingerprint image, and determines a region to extract the characteristic information based on contrast information on each of the images and the like. For example, the extraction unit 132 compares the first fingerprint image with the second fingerprint image, and determines regions in the respective images where the contrast in the region in one image is higher than the contrast in the corresponding region in the other image, or in other words, the regions where the fingerprint pattern or the like constituting the characteristic information is clearly imaged. Then, the extraction unit 132 extracts the characteristic information from the determined regions. The extraction unit 132 divides each of the first fingerprint image and the second fingerprint image into multiple sections and compares a contrast value in a certain section of one of the images with a contrast value in a section of the other image located at the same position. Thus, the extraction unit 132 extracts the sections in the respective images, each of which has a higher contract value than that in the corresponding section in the other image. Alternatively, the extraction unit 132 may generate a composite image of the first fingerprint image and the second fingerprint image and extract the characteristic information from the composite image. In this way, it is possible to extract the characteristic information to be used for authentication from a wider range as compared with the case of using the fingerprint image that is obtained by imaging the reflected light from the finger F originating from the illumination light projected from the single direction of projection onto the finger F.

Next, the authentication unit 133 performs authentication based on the characteristic information extracted by the extraction unit 132 (step S26). For example, the same processing as the above-described step S14 is carried out in step S26.

In step S25, the extraction unit 132 may further compare the first fingerprint image with the second fingerprint image so as to determine whether the imaged finger is the true finger of the actual living body or the false finger either printed or displayed on a flat surface. For example, the extraction unit 132 compares the first fingerprint image with the second fingerprint image, and determines that the finger is the false finger when the first fingerprint image and the second fingerprint image have a degree of similarity greater than or equal to a predetermined value or determines that the finger is the finger of the living body when the first fingerprint image and the second fingerprint image have the degree of similarity less than the predetermined value. The extraction unit 132 outputs information to notify the authenticatee of a result of determination, for example.

### 3. Modified Example

Next, a contactless authentication system according to a modified example of Embodiment 2 will be described. In Embodiment 2, the multiple illumination apparatuses project the illumination light, thereby irradiating the finger with the illumination light in the directions of projections different from each other. On the other hand, in the modified example of Embodiment 2, the finger is irradiated with the illumination light in the directions of projections different from each other by causing the illumination apparatus to change the direction of projection of the illumination light.

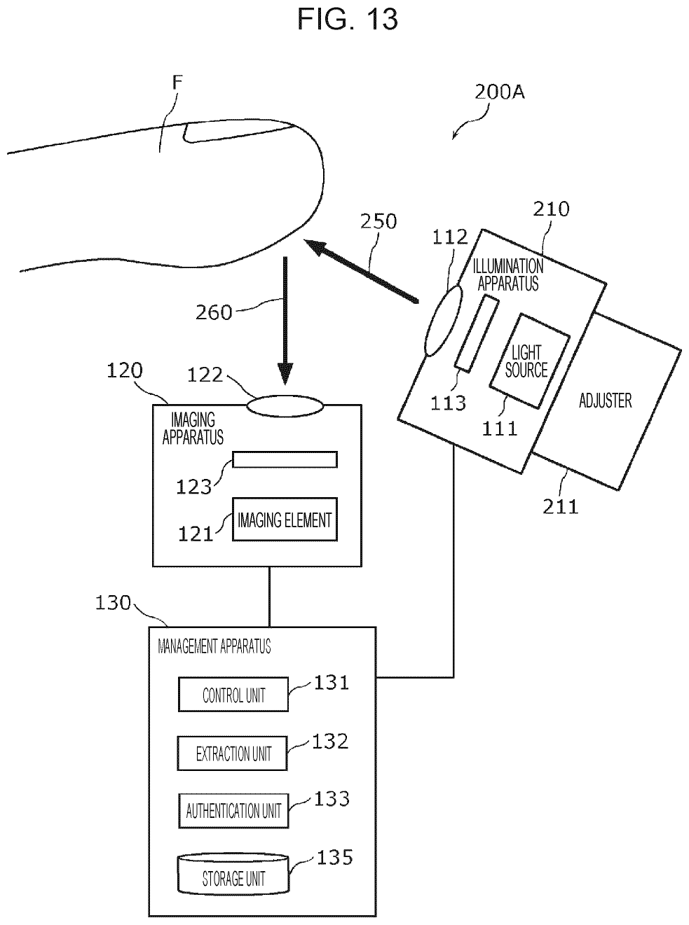

FIG. 13 is a block diagram illustrating a schematic configuration of a contactless authentication system 200A according to the modified example. As illustrated in FIG. 13, in comparison with the contactless authentication system 100 according to Embodiment 1, the contactless authentication system 200A is different in that an illumination apparatus 210 is provided instead of the illumination apparatus 110. In other words, the contactless authentication system 200A according to the modified example of Embodiment 2 includes the illumination apparatus 210, the imaging apparatus 120, and the management apparatus 130.

The illumination apparatus 210 is the apparatus that can change the direction of projection of illumination light 250 to be projected. In addition to the light source 111, the illumination optical system 112, and the optical filter 113 as with the illumination apparatus 110, the illumination apparatus 210 further includes an adjuster 211 for adjusting the direction of projection of the illumination light 250 onto the finger F.

The adjuster 211 changes the direction of projection of the illumination light 250 onto the finger F. For example, the adjuster 211 includes a mechanism for making the illumination apparatus 210 movable. Thus, the illumination apparatus 210 moves in such a way as to change the direction of projection of the illumination light 250 onto the finger F. Alternatively, the adjuster 211 may include a mechanism for making the illumination optical system 112 movable, for example. Accordingly, the direction of projection of the illumination light 250 is changed by causing the illumination optical system 112 to change an optical path of the light emitted from the light source 111. The adjuster 211 is formed from a drive device such as an actuator or a motor connected to a casing of the illumination apparatus 210 or to the illumination optical system 112, for example. Alternatively, the adjuster 211 may be formed from a set of a movable shaft and a supporting member, a slider, and the like for changing the direction of projection of the illumination light 250 by hand.

Regarding an operation of the contactless authentication system 200A, the illumination apparatus 210 projects the illumination light 250 as the first illumination light onto the finger F in step S21 of a flowchart illustrated in FIG. 12. Meanwhile, in step S23, the illumination apparatus 210 projects the illumination light 250 as second illumination light having the direction of projection different from that of the first illumination light by causing the adjuster 211 to change the direction of projection of the illumination light 250. The adjuster 211 changes the direction of projection of the illumination light 250 based on control by the control unit 131 of the management apparatus 130 or an operation of a user, for example. Thus, the imaging apparatus 120 images reflected light 260 and obtains the first fingerprint image and the second fingerprint image. For the rest of the steps, the contactless authentication system 200A carries out the same operations as those by the contactless authentication system 200.

### Embodiment 3

Next, a contactless authentication system according to Embodiment 3 will be described. Embodiment 3 will describe an example of a contactless authentication system including an illumination apparatus provided with a modulated illumination function and an imaging apparatus provided with a sensitivity modulating function. In the following description of Embodiment 3, a description will be given mainly on features different from those of Embodiments 1 and 2, and explanations of common features will be simplified or omitted.

### 1. Configuration of Contactless Authentication System

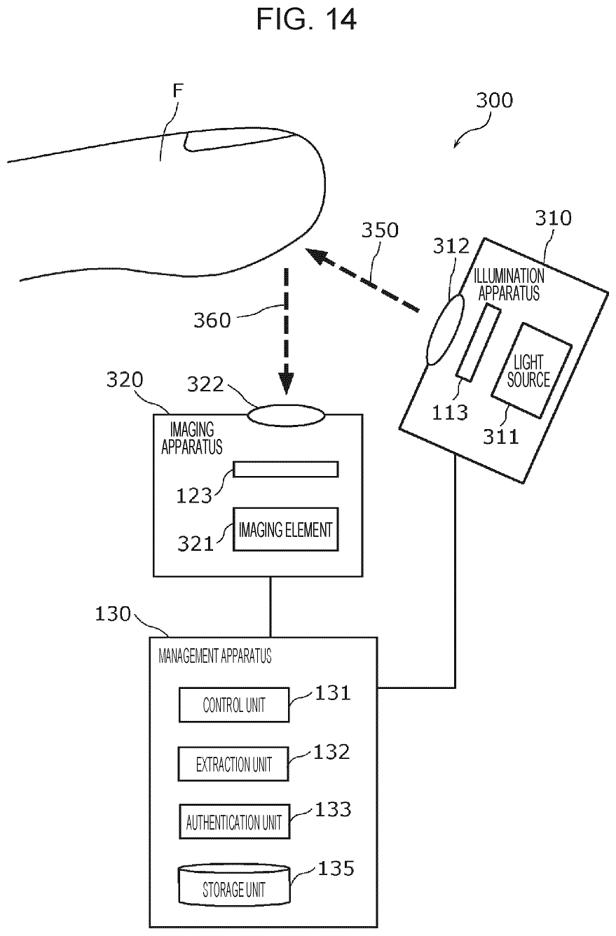

FIG. 14 is a block diagram illustrating a schematic configuration of a contactless authentication system 300 according to the present embodiment. As illustrated in FIG. 14, in comparison with the contactless authentication system 100 according to Embodiment 1, the contactless authentication system 300 is different in that an illumination apparatus 310 that cyclically changes an emission intensity of illumination light 350 and an imaging apparatus 320 that cyclically changes sensitivity are provided instead of the illumination apparatus 110 and the imaging apparatus 120. In other words, the contactless authentication system 300 includes the illumination apparatus 310, the imaging apparatus 320, and the management apparatus 130. In the present specification, an act of cyclically changing the emission intensity or the sensitivity may be described as modulation as appropriate.

The illumination apparatus 310 includes a light source 311, an illumination optical system 312, and the optical filter 113. Meanwhile, the imaging apparatus 320 includes an imaging element 321, an imaging optical system 322, and the optical filter 123. Requirements for the wavelength of illumination light 350 to be projected by the illumination apparatus 310, the wavelength range of light to be imaged by the imaging apparatus 320 as the main imaging component, and so forth are basically the same as those used in the contactless authentication system 100 according to Embodiment 1.

The illumination apparatus 310 has a function to cyclically change the emission intensity of the illumination light 350 to be projected. This function may be realized, for example, by applying a light emitting element such as a laser diode or a light emitting diode configured to adjust an amount of light by current control or voltage control and a power source configured to cyclically change either a current or a voltage in a repeated manner to the light source 311. Alternatively, the light source 311 may be a light source such as a pulse laser, which is configured to emit light having an intensity that changes temporally and cyclically. Meanwhile, this function may be realized by providing the illumination optical system 312 of the illumination apparatus 310 with a shutter or a chopping blade which can cyclically repeat opening and closing, thereby cyclically changing the emission intensity of the illumination light 350 to be projected onto the finger F being the subject. In the meantime, the illumination apparatus 310 may include an acousto-optical element or an electro-optical modulator and perform intensity modulation of the illumination light 350 by using any of these devices.

The illumination apparatus 310 may change the intensity of the illumination light 350 continuously such as an offset sinusoidal wave, or change the intensity of the illumination light 350 discretely such as a pulse train.

Since the emission intensity of the illumination light 350 is cyclically changed, an emission intensity of reflected light 360 from the finger F originating from the illumination light 350 is also changed at the same cycle. The imaging apparatus 320 images the reflected light 360.

The imaging apparatus 320 has a function to cyclically change its sensitivity in response to the cyclical change of the illumination light 350 in an exposure period. Here, the exposure period means a period from a point when the imaging element 321 resets the accumulated signal charges and starts accumulating the signal charges to a point when the imaging element 321 starts reading out the signal charges. A cycle of change in sensitivity of the imaging apparatus 320 is the same as a cycle of change in emission intensity of the illumination light 350, for example. Here, when both the change in intensity of the illumination light 350 and the change in sensitivity of the imaging apparatus are in the form of discrete pulses, one of the cycle may be equal to an integral multiple of the other cycle.

An image intensifier camera (an ICCD camera) is an example of the imaging apparatus 320 provided with a function to modify sensitivity at a high speed. The ICCD camera multiplies electrons, which are generated by incidence of light on a light receiving surface, by using a multichannel plate, and causes the multiplied electrons to collide with a fluorescent screen. Hence, the camera images fluorescent light generated on the screen. In this instance, it is possible to cyclically change the sensitivity by cyclically changing a voltage to be applied to the multichannel plate.

In the meantime, examples of the imaging element 321 for realizing the imaging apparatus 320 having the function to modulate the sensitivity at a high speed include the laminated image sensor and a charge distribution element.

The laminated image sensor is the imaging element having the structure to sandwich the photoelectric conversion layer between the counter electrode and the pixel electrodes as illustrated in FIG. 7. The sensitivity of the laminated image sensor depends on the electric potential difference between the transparent electrode and the pixel electrode, or so-called a bias voltage. The sensitivity can be set substantially equal to zero by setting the bias voltage less than or equal to a predetermined threshold. On the other hand, even when the bias voltage is greater than or equal to the predetermined threshold, the sensitivity varies with the bias voltage, for example. A detailed description is found in, for example, Japanese Unexamined Patent Application Publication No. 2017-208812 filed by the inventor of the present application regarding the aforementioned sensitivity modulation in the laminated image sensor.

The charge distribution element is an imaging element including two or more charge collectors, or including one or more charge collectors and a charge discarder for the photoelectric conversion region in each pixel. Examples of the charge distribution element include a multitap CCD and a transfer modulation type laminated image sensor.

A detailed description is found in Japanese Patent No. 4235729 regarding the multitap CCD. Meanwhile, detailed descriptions are found in International Publication No. WO 2021/176876 filed by the inventor of the present specification and in US Patent Application Publication No. 2019/0252455 regarding the transfer modulation type laminated image sensor.

In the case of the charge distribution element, when the element has a configuration to provide each photoelectric conversion region with two or more charge collectors, the element can simultaneously obtain two fingerprint images that represent a result of imaging while modulating two types of sensitivity having phases different from each other. As described later, according to the present embodiment, it is possible to remove the ambient light effectively by obtaining both an imaging result of imaging while changing the sensitivity higher at a high phase of the intensity of the illumination light 350 and an imaging result of imaging while changing the sensitivity higher at a low phase of the intensity of the illumination light 350. By using the charge distribution element as the imaging element 321 as described above, it is possible to obtain the two imaging results as mentioned above simultaneously, thereby effectively removing the ambient light.

Meanwhile, imaging apparatus 320 may be cyclically change the sensitivity by providing the imaging optical system 322 with a shutter or a chopper, which physically and cyclically blocks the light that is incident on the imaging element 321, for example.

The contactless authentication system 300 switches a correlation between the phase of the change in intensity of the illumination light 350 and the phase of change in sensitivity of the imaging apparatus 320 into two states by control of the control unit 131, for example. To be more precise, the contactless authentication system 300 switches between a case where the sensitivity of the imaging apparatus 320 reaches the high phase at the high phase of the emission intensity of the illumination light 350 and a case where the sensitivity of the imaging apparatus 320 reaches the high phase at the low phase of the emission intensity of the illumination light 350.

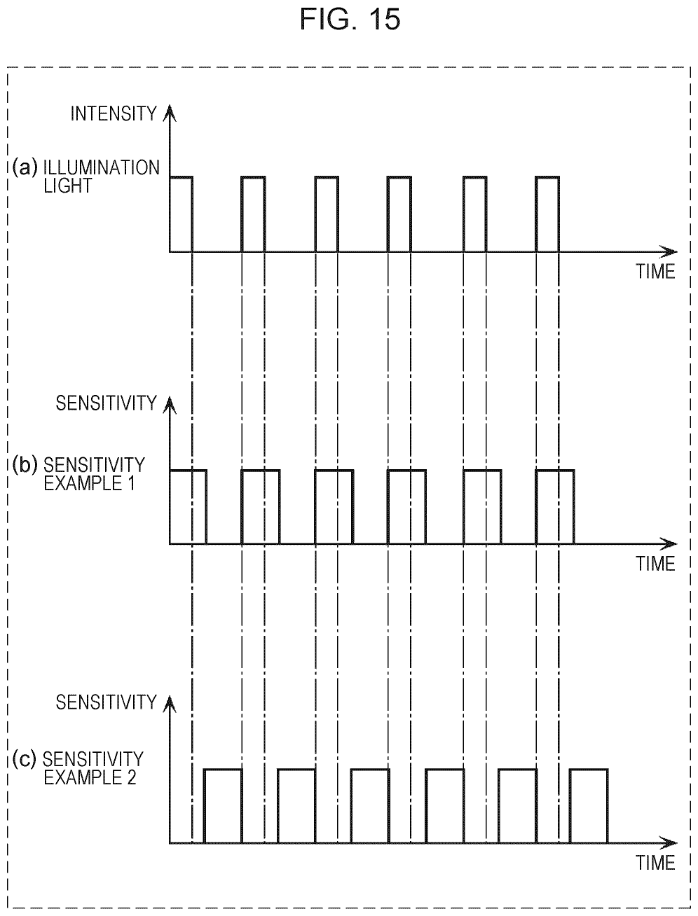

FIG. 15 is a diagram illustrating examples of the change in emission intensity of the illumination light 350 and of the change in sensitivity of the imaging apparatus 320. Portion (a) of FIG. 15 illustrates an example of the change in emission intensity of the illumination light 350, while Portion (b) and Portion (c) of FIG. 15 illustrate sensitivity example 1 and sensitivity example 2, respectively, which are examples of the change in sensitivity of the imaging apparatus 320. When the illumination light 350 illustrated in Portion (a) of FIG. 15 is projected, for example, the contactless authentication system 300 switches between the case of imaging at the sensitivity of the sensitivity example 1 and the case of imaging at the sensitivity of the sensitivity example 2 by the imaging apparatus 320. Each period at the high sensitivity of the imaging apparatus 320 in the sensitivity example 1 and each period at the high sensitivity of the imaging apparatus 320 in the sensitivity example 2 have the same length. Moreover, the sensitivity at the high phase of the sensitivity of the imaging apparatus 320 in the sensitivity example 1 and the sensitivity at the high phase of the sensitivity of the imaging apparatus 320 in the sensitivity example 2 have the same level. Meanwhile, in FIG. 15, each period at the high emission intensity of the illumination light 350 is shorter than each period at the high sensitivity of the imaging apparatus 320. However, the period at the high emission intensity of the illumination light 350 may be equal to the period at the high sensitivity of the imaging apparatus 320.

The above-described control of the emission intensity and the sensitivity may be realized, for example, by adopting a configuration in which a cyclic signal generating device such as a function generator not illustrated in FIG. 14 is provided to the contactless authentication system 300 in addition to the illumination apparatus 310 and the imaging apparatus 320, and both the illumination apparatus 310 and the imaging apparatus 320 receive output from the cyclic signal generation device. Meanwhile, the above-described control of the emission intensity and the sensitivity may be realized by causing the control unit 131 to output cyclic signals to the illumination apparatus 310 and the imaging apparatus 320. Alternatively, a circuit and the like having a function to output the aforementioned cyclic signals may be included in the illumination apparatus 310 or the imaging apparatus 320.

### 2. Operation Examples of Contactless Authentication System

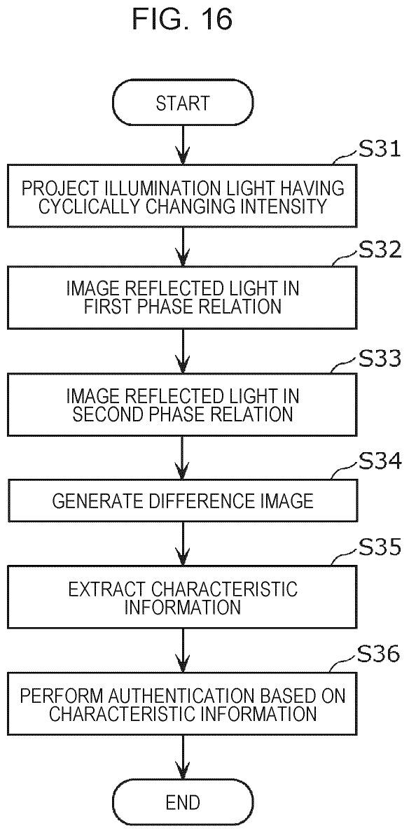

Next, operations of the contactless authentication system 300 according to the present embodiment will be described. FIG. 16 is a flowchart illustrating an operation example of the contactless authentication system 300 according to the present embodiment.

As illustrated in FIG. 16, the illumination apparatus 310 projects the illumination light 350 having the cyclically changing intensity onto the finger F to begin with (step S31). The illumination apparatus 310 irradiates the finger F with the illumination light 350 that has the emission intensity as illustrated in Part (a) of FIG. 15, for example.

Next, the imaging apparatus 320 images the reflected light 360 generated by reflection of the illumination light 350 from the finger F after being projected onto the finger F in the state where the phase of the change in emission intensity of the illumination light 350 and the phase of the change in sensitivity of the imaging apparatus 320 satisfy a first phase relation (step S32). The imaging apparatus 320 changes the sensitivity of the imaging apparatus 320 at the same cycle as that of the change in emission intensity of the illumination light 350 such that the phase of the change in emission intensity of the illumination light 350 and the phase of the change in sensitivity of the imaging apparatus 320 satisfy a phase relation in which the sensitivity of the imaging apparatus 320 is set to the high phase at the high phase of the emission intensity of the illumination light 350 as illustrated in Part (a) and Part (b) of FIG. 15, for example. In this way, the imaging apparatus 320 obtains a third fingerprint image being the imaging result as the authentication information. The imaging apparatus 320 outputs the obtained third fingerprint image to the management apparatus 130, for example. The extraction unit 132 of the management apparatus 130 obtains the third fingerprint image from the imaging apparatus 320, and stores the image in the storage unit 135.

Then, the imaging apparatus 320 images the reflected light 360 generated by reflection of the illumination light 350 from the finger F after being projected onto the finger F in the state where the phase of the change in emission intensity of the illumination light 350 and the phase of the change in sensitivity of the imaging apparatus 320 satisfy a second phase relation (step S33). The imaging apparatus 320 changes the sensitivity of the imaging apparatus 320 at the same cycle as that of the change in emission intensity of the illumination light 350 such that the phase of the change in emission intensity of the illumination light 350 and the phase of the change in sensitivity of the imaging apparatus 320 satisfy a phase relation in which the sensitivity of the imaging apparatus 320 is set to the high phase at the low phase of the emission intensity of the illumination light 350 as illustrated in Part (a) and Part (c) of FIG. 15, for example. In this way, the imaging apparatus 320 obtains a fourth fingerprint image being the imaging result as the authentication information. The imaging apparatus 320 outputs the obtained fourth fingerprint image to the management apparatus 130, for example. The extraction unit 132 of the management apparatus 130 obtains the fourth fingerprint image from the imaging apparatus 320, and stores the image in the storage unit 135.

Next, the extraction unit 132 generates a difference image between the third fingerprint image and the fourth fingerprint image stored in the storage unit 135 (step S34). The extraction unit 132 generates the difference image by subtracting the fourth fingerprint image from the third fingerprint image, for example. To be more precise, the extraction unit 132 generates the difference image by calculating differences in pixels values of respective pixels in the third fingerprint image and the fourth fingerprint image, for instance.

Then, the extraction unit 132 extracts the characteristic information to be used for authentication from the generated difference image (step S35). The same processing as the above-described step S13 is carried out in step S35 except that the difference image is used instead of the fingerprint image.

Next, the authentication unit 133 performs authentication based on the characteristic information extracted by the extraction unit 132 (step S36). The same processing as the above-described step S14 is carried out in step S36, for example.

Accordingly, the third fingerprint image and the fourth fingerprint image include influence of the light other than the illumination light 350 such as the solar light and indoor illumination light, or so-called the ambient light besides the illumination light 350. The ambient light included in the third fingerprint image is nearly equal to that included in the fourth fingerprint image when the periods of the high sensitivity of the imaging apparatus 320 are equal between step S32 and step S33. Accordingly, the component of the ambient light is offset in the difference image between the third fingerprint image and the fourth fingerprint image. Even when the periods of the high sensitivity of the imaging apparatus 320 vary between these steps, it is possible to subtract the component of the ambient light by adopting a correction coefficient corresponding to the difference in length between the periods in the process of generating the difference image.

Meanwhile, the third fingerprint image represents the imaging result in the case where the sensitivity of the imaging apparatus 320 reaches the high phase when the emission intensity of the illumination light 350 is set to the high phase. Accordingly, the component of the reflected light 360 from the finger F originating from the illumination light 350 contained in the third fingerprint image is more than that contained in the fourth fingerprint image. Because the third fingerprint image represents the imaging result in the case where the sensitivity of the imaging apparatus 320 reaches the high phase when the emission intensity of the illumination light 350 is set to the high phase, whereas the fourth fingerprint image represents the imaging result in the case where the sensitivity of the imaging apparatus 320 reaches the high phase when the emission intensity of the illumination light 350 is set to the low phase. As a consequence, in the difference image, the ambient light component is subtracted from the third fingerprint image, and the reflected light 360 component is left therein. Thus, the difference image contains the information deriving from the reflected light 360 in the state of reducing the influence of the ambient light. In this way, the contrast and other factors in the difference image originating from the shape of the fingerprint are increased, so that the information to be extracted can be extracted easily so as to improve accuracy of authentication.

Note that the above-described operation example is a mere example. Similar effects are available by shooting the fingerprint image by using the two phase relations between the change in intensity of the illumination light and the change in sensitivity in which the amounts of the component of the reflected light 360 contained in the fingerprint images are different from each other. For example, instead of changing the phase of the sensitivity of the imaging apparatus 320, the fingerprint image may be obtained by imaging while using the different phase relations by changing the phase of the emission intensity of the illumination light 350. In the meantime, the cycle of the change in emission intensity of the illumination light 350 and the cycle of the change in sensitivity of the imaging apparatus 320 do not have to be constant.

Meanwhile, step S32 and step S33 can be carried out at the same time when the imaging element 321 is the charge distribution element. Accordingly, it is possible to shorten the imaging time and to reduce variations of the ambient light and of the subject between moments of shooting the two fingerprint images, thereby effectively removing the ambient light.

### OTHER EMBODIMENTS

The contactless authentication system according to the present disclosure has been described above based on the embodiments and a modified example. It is to be noted, however, that the present disclosure is not limited to the embodiments and the modified example.

For example, in addition to the configuration to image the reflected light while defining the wavelength range greater than or equal to 1380 nm as the main imaging component, the imaging apparatus may also image the reflected light while defining the wavelength range less than 1380 nm as the main imaging component. In this case, for example, the imaging apparatus includes multiple optical filters having different transmission wavelength ranges, and images the reflected light while defining the different wavelength ranges as the main imaging component by switching the optical filters. Alternatively, the imaging element of the imaging apparatus may include pixels for imaging light having the wavelength greater than or equal to 1380 nm and pixels for imaging light having the wavelength less than 1380 nm. In the meantime, the contactless authentication system may be provided with multiple imaging apparatuses including the imaging apparatus that images the reflected light while defining the wavelength range greater than or equal to 1380 nm as the main imaging component and the imaging apparatus that images the reflected light while defining the wavelength range less than 1380 nm as the main imaging component.

When the subject is an actual finger, the contrast of the fingerprint image obtained by imaging while defining the wavelength range greater than or equal to 1380 nm as the main imaging component is higher than the contrast of the fingerprint image obtained by imaging while defining the wavelength range less than 1380 nm as the main imaging component. As described above, this is attributed to the variation of the ratio between the surface reflected light and the scatter-reflected light originating from the subsurface scattering light with the wavelength due to spectral absorption characteristics of tissues constituting the finger.

On the other hand, in the case of a false finger made of a resin or the like or in the case of a finger image printed on paper or a finger image displayed on a display unit, a relation between the contrast of the fingerprint image obtained by imaging while defining the wavelength range greater than or equal to 1380 nm as the main imaging component and the contrast of the fingerprint image obtained by imaging while defining the wavelength range less than 1380 nm as the main imaging component may be different from that in the case of the actual finger. This is because the spectral absorption characteristics of the false finger may be different from those of the actual finger. For example, the false finger has a smaller degree of absorption by the moisture than that of the actual finger. Accordingly, the difference in contrast between the two fingerprint images mentioned above in the case of the false finger is smaller than the difference in contrast between the two fingerprint images mentioned above in the case of the actual finger. As a consequence, the false finger may probably be detected by using the relation of the contrast in the fingerprint images obtained by imaging while defining the two different types of the wavelength ranges as the main imaging components. For example, in addition to the individual authentication, the authentication unit of the management apparatus may also determine whether or not the subject is the false finger by obtaining the above-mentioned two fingerprint images and comparing the two fingerprint images with each other.

In the meantime, for example, the illumination apparatus may have functions to irradiate the finger with linear illumination light, and to sequentially shift the irradiation position. As compared with the case of projecting the illumination light in a planar form, this mode can increase a density of the illumination light. Thus, the imaging apparatus can obtain an image at a high signal-noise ratio. Meanwhile, when the linear light is projected onto the three-dimensional finger, a shape of the irradiated region takes on a curved line. Using this phenomenon, it is possible to identify the false finger printed on a flat surface or displayed on a flat display device. The irradiation position can be changed by using a galvano mirror, for example.

For example, the subject is the finger in the embodiments and the modified example described above. Instead, the subject may be a palm, or both the finger and the palm, for example.

The contactless authentication system is realized by using the multiple apparatuses in the embodiments and the modified example described above. Instead, the contactless authentication system may be realized as a single apparatus, for example. In the meantime, when the contactless authentication system is realized by using the multiple apparatuses, the constituents provided to the contactless authentication system discussed in the embodiments and the modified example described above may be allocated to the apparatuses in any way.

Meanwhile, the contactless authentication system does not always have to include all of the constituents discussed in the embodiments and the modified example described above. The contactless authentication system may include only the constituents required for implementing an intended operation.

For example, the contactless authentication system may include a communication unit. Meanwhile, the management apparatus may be an external device such as a smartphone owned by a user, a dedicated device brought in by the user, or a cloud server. Here, the contactless authentication system may conduct authentication by communicating with the external device while using the communication unit.

In the above-described embodiments, the processing to be executed by a particular one of the processing units may be executed by a different processing unit. Meanwhile, the order of processing procedures may be changed or multiple processing procedures may be carried out in parallel.

In the above-described embodiments, the respective constituents may be implemented by executing software programs suitable for the respective constituents. The respective constituents may be implemented by causing a program execution unit such as a CPU or a processor to read a software program stored in a storage medium such as a hard disk drive or a semiconductor memory and to execute the program.

Alternatively, the respective constituents may be implemented by using hardware. Each of the constituents may be a circuit (or an integrated circuit). These circuits may constitute a single circuit as a whole, or may be provided as independent circuits. Moreover, each of these circuits may be a general-purpose circuit or a dedicated circuit.

Moreover, each of the general and specific aspects of the present disclosure may be realized by using a system, an apparatus, a method, an integrated circuit, a computer program, and a computer-readable storage medium such as a CD-ROM. Alternatively, such an aspect may also be realized by a desired combination of any of a system, an apparatus, a method, an integrated circuit, a computer program, and a storage medium.

For example, the present disclosure may be realized as the contactless authentication system according to any of the above-described embodiments or realized as a program for causing a computer to implement an authentication method to be carried out by the processing units. The present disclosure may also be realized in the form of a non-transitory computer-readable storage medium storing the aforementioned program.

In addition, various modifications conceived by those skilled in the art may be applied to the embodiments and examples thereof, and other modes may also be constructed by combining selected constituents that are discussed in the embodiments and the examples. All of these modifications and combinations are also encompassed by the scope of the present disclosure within the range not departing from the gist of the present disclosure.

The contactless authentication system and the authentication method according to the present disclosure are applicable to entrance management of a building and authentication at a gate in an airport, for example.

What is claimed is:

1. A contactless authentication system comprising:
at least one illumination apparatus that projects illumination light onto a portion of a hand, the portion being not in contact of an object, the illumination light containing a light component in a wavelength range greater than or equal to 1380 nm; and
an imaging apparatus that obtains at least one selected from the group consisting of a fingerprint image and a palm print image as authentication information by imaging the light component in the wavelength range in reflected light generated by reflection of the illumination light from the portion of the hand.

2. The contactless authentication system according to claim 1, wherein the authentication information includes information indicating a position of a sweat pore.

3. The contactless authentication system according to claim 1, wherein
the imaging apparatus includes a photoelectric conversion layer, and
sensitivity of the photoelectric conversion layer has a peak in the wavelength range.

4. The contactless authentication system according to claim 3, wherein the photoelectric conversion layer includes a quantum dot.

5. The contactless authentication system according to claim 3, wherein the photoelectric conversion layer includes a semiconducting carbon nanotube.

6. The contactless authentication system according to claim 1, wherein the light component to be imaged by the imaging apparatus contains a wavelength at which solar light is significantly attenuated on a ground surface.

7. The contactless authentication system according to claim 1, wherein
the imaging apparatus includes an optical filter, and
a transmittance of the optical filter with respect to light having a wavelength less than 1380 nm is lower than a transmittance of the optical filter with respect to light having a wavelength greater than or equal to 1380 nm.

8. The contactless authentication system according to claim 1, wherein
the at least one illumination apparatus cyclically changes an emission intensity of the illumination light, and
the imaging apparatus cyclically changes sensitivity of the imaging apparatus in response to change in the emission intensity of the illumination light.

9. The contactless authentication system according to claim 1, wherein
the at least one illumination apparatus projects the illumination light onto the hand in a first direction and in a second direction different from the first direction, and
the imaging apparatus images the reflected light originating from the illumination light projected onto the hand in the first direction and the reflected light originating from the illumination light projected onto the hand in the second direction.

10. The contactless authentication system according to claim 9, wherein
the at least one illumination apparatus includes
a first illumination apparatus that projects the illumination light onto the hand in the first direction, and
a second illumination apparatus that projects the illumination light onto the hand in the second direction, and
timing to project the illumination light from the first illumination apparatus onto the hand is different from timing to project the illumination light from the second illumination apparatus onto the hand.

11. The contactless authentication system according to claim 9, wherein
the at least one illumination apparatus includes an adjuster that changes a direction of projection of the illumination light onto the hand, and
the at least one illumination apparatus projects the illumination light onto the hand in the first direction and the second direction by using the adjuster.

12. The contactless authentication system according to claim 1, wherein the light component imaged by the imaging apparatus is a light component in the reflected light in a wavelength range greater than or equal to 1380 nm and less than 2500 nm.

13. An authentication method comprising:
projecting illumination light onto a portion of a hand, the portion being not in contact of an object, the illumination light containing a light component in a wavelength range greater than or equal to 1380 nm; and
obtaining at least one selected from the group consisting of a fingerprint image and a palm print image as authentication information by imaging the light component in the wavelength range in reflected light generated by reflection of the illumination light from the portion of the hand.
